(19) United States
(12) Patent Application Publication
(10) Pub. No.: US 2021/0361588 A1
(43) Pub. Date: Nov. 25, 2021

(54) COMPOSITIONS FOR THE TREATMENT OF A DISEASE OF THE URINARY TRACT AND TREATMENT OF A DISEASE INVOLVING THE INTRACELLULAR DELIVERY OF THE PARTICLE OR A MEDICAMENT CONTAINED THEREIN

(71) Applicant: UCL BUSINESS LTD, London (GB)

(72) Inventors: Eleanor Phoebe Jane STRIDE, London (GB); Ann SAVELL, London (GB); Jennifer ROHN, London (GB); Wai Keith LAU, London (GB); Mohan EDIRISINGHE, London (GB)

(21) Appl. No.: 16/962,948

(22) PCT Filed: Jan. 18, 2019
(86) PCT No.: PCT/GB2019/050152

§ 371 Date: Jul. 17, 2020

(30) Foreign Application Priority Data
Jan. 19, 2018 (GB) 1800912.6

(51) Int. Cl. A61K 9/50; A61K 45/06

(52) U.S. Cl. CPC A61K 9/5031; A61K 45/06; A61K 9/5089

(57) ABSTRACT

Described herein is a composition comprising a particle comprising a biodegradable and hydrolysable polymer, wherein a medicament for the treatment of a disease of the urinary tract is dispersed in the polymer, wherein the particle has a dimension of from 1 μm to 30 μm. Also described herein is a composition for the treatment of a disease, the composition comprising a particle comprising a biodegradable and hydrolysable polymer, wherein a medicament is dispersed in the polymer, wherein the particle has a dimension of from 1 μm to 30 μm, wherein the composition is for use in a method for treating the disease and the method involves the intracellular delivery of the particle or the medicament from the particle. Also described herein is a composition comprising a particle comprising a biodegradable and hydrolysable polymer, wherein a medicament is dispersed in the polymer, wherein the particle has a dimension of from 1 μm to 30 μm, wherein the composition is for use in a method for treating bacteria and optionally the method involves the intracellular delivery of the particle or the medicament from the particle.

### BACKGROUND

Diseases of the urinary tract include those that affect the bladder, kidney or ureter. Examples of such diseases include urinary tract infections (UTI), cancer of the bladder, and/or kidney and over-active bladder, among others.

At 150 million cases per annum, urinary tract infection (UTI) is one of the most common infectious diseases globally, and is the top infection amongst the elderly population. Its frequency results in a massive economic and healthcare burden in society, with half of all women experiencing one in their lifetimes. Although uncomplicated UTI in otherwise healthy people can be self-limiting or treatable with traditional antibiotics, UTI is more problematic in a subset of patients, including the elderly, pregnant women, people with multiple sclerosis, spinal injuries or renal transplantation, and those needing urinary catheters. Uncontrolled UTI can lead to life-threatening kidney infection and sepsis, and UTI is one of the most common hospital-acquired infections, leading to an increase in bed-days and associated costs for healthcare.

Arguably one of the most problematic aspects of UTI is its tendency to recur, even in otherwise healthy people. Thus, at least 25% of all uncomplicated UTIs will recur within the same year, and a further subset of those initially infected will experience a second relapse that year, with some unfortunate people suffering from repeated recurrences. There is also growing evidence that chronic or recurrent, often lower-level UTI can cause lower urinary tract symptoms (LUTS) or “overactive bladder”, especially in the elderly, which also has a great economic and healthcare impact.

The reasons for recurrence and chronicity are not yet entirely clear, but several factors probably play a role, including host genetic background, evasion strategies of the bacteria, and inadequacy of standard oral antibiotic therapy in the face of those strategies. For example, it's well known that several uropathogens (e.g. pathogens of the urinary tract) can invade and lie sequestered within bladder cells in protected reservoirs, and they also have the capability of forming treatment-resistant biofilms. These issues also affect other diseases caused by any bacterial infection, especially bacterial infections which occur intracellularly.

Standard oral drug therapy has many drawbacks. Oral drugs are often poorly absorbed, require prolonged exposure and require a high dosage of active agent for therapeutic effect, often leading to side effects. Furthermore, oral antibiotic therapy has perceived undesirability which can lead to compliance issues, and the threat of exacerbating a growing global antimicrobial resistance (AMR) crisis. AMR is a particular problem in uropathogenic bacteria worldwide.

In the case of UTI, and other bacterial infections, there are three additional drawbacks to antibiotics. First, the systemic dose required to achieve antimicrobial killing (e.g. in the bladder) can be quite high, so the entire system must be exposed to high levels of the drug in order to maintain a high dose.

The second main drawback, in the case of UTI and other bacterial infections, is that many antibiotics are cell-impermeant, so they would not be able to access intracellular reservoirs. One could argue that direct intravesical treatments of cell-permeant antibiotics could circumvent this problem, but even cell-permeant antibiotics may not accumulate to high enough levels within cells in the bladder wall, as it's known that free diffusion is markedly inefficient compared with directed delivery. Indeed, antibiotic bladder instillation is very rare in the clinic and the literature suggests that, aside from gentamicin in the case of intermittent catheter use, such treatments don't result in cures for UTI. This lack of success has also been demonstrated with installations of nitrofurans.

Third, biofilms are naturally resistant to free antibiotics due to a variety of mechanisms, including both a physical barrier to drug diffusion, as well as lack of active growth and division. These molecular pathways are the normal targets for most antibiotics. Uropathogenic bacteria, among other pathogenic bacteria, are known to form biofilms, so this behaviour could retard standard treatments for UTI and other bacterial infections, and would thwart direct installations of free antibiotics (e.g. into the bladder).

A previous invention (Labbaf et al, 2013) developed a polymeric capsule which demonstrated bacterial killing in shaking broth cultures, however whilst the particles could penetrate cells in culture, (Labbaf et al, 2013) the penetration efficiency was very low.

### SUMMARY OF THE INVENTION

In a first aspect, the present invention provides a composition comprising a particle comprising a biodegradable and hydrolysable polymer, wherein a medicament for the treatment of a disease of the urinary tract is dispersed in the polymer, wherein the particle has a dimension of from 1 μm to 30 μm. The composition may be used in a method for intracellular delivery of the medicament. In an embodiment, the particle is delivered via a synthetic conduit to the urinary tract for the treatment. The composition may comprise a plurality of the particles comprising the biodegradable and hydrolysable polymer. The composition may be producible by a method of the fifth aspect.

In a second aspect, the present invention provides a composition for the treatment of a disease, the composition comprising a particle comprising a biodegradable and hydrolysable polymer, wherein a medicament is dispersed in the polymer, wherein the particle has a dimension of from 1 μm to 30 μm, wherein the composition is for use in a method for treating the disease and the method involves the intracellular delivery of the particle or the medicament from the particle.

In a third aspect, there is provided a method for the treatment of a disease of the urinary tract, the method comprising administering to the urinary tract of a host: a composition comprising a particle comprising a biodegradable and hydrolysable polymer, wherein a medicament for the treatment of a disease of the urinary tract is dispersed in the polymer, wherein the particle has a dimension of from 1 μm to 30 μm.

In a fourth aspect, there is provided a method for the treatment of a disease, the method involving the intracellular delivery of a composition comprising a particle comprising a biodegradable and hydrolysable polymer, wherein a medicament is dispersed in the polymer, wherein the particle has a dimension of from 1 μm to 30 μm.

In a fourth aspect, there is also provided a method for the treatment of a disease, the method involving the intracellular delivery of a composition or a medicament from the composition comprising a particle comprising a biodegradable and hydrolysable polymer, wherein the medicament is dispersed in the polymer, wherein the particle has a dimension of from 1 μm to 30 μm.

In a fifth aspect, there is provided a process for producing particles comprising a biodegradable and hydrolysable polymer, the process comprising:
(i) providing an electrohydrodynamic device comprising at least two concentrically arranged, spaced apart hollow needles, the needles together defining a core channel, and an outer concentrically disposed tubular channel; and a means for applying a voltage to the needles
(ii) passing fluid mediums through the hollow core channel, and the outer concentrically disposed tubular channel, wherein at least one of the fluid mediums in one of the channels has therein a biodegradable and hydrolysable polymer and a medicament, optionally for the treatment of a disease of the urinary tract,
(iii) applying a voltage to the needles, such that, on leaving the needles, the particles comprising the biodegradable and hydrolysable polymer are formed, wherein the medicament, optionally for the treatment of a disease of the urinary tract, is dispersed in the polymer, and at least some of the particles have a dimension of from 1 μm to 30 μm.

The present inventors have developed an improved composition that allows penetration activity that is robust and efficient enough to introduce a high level of a medicament inside cells (considerably higher than concentrations achievable by free diffusion). In addition, the particles of the composition have been shown to penetrate multiple layers of cells and is also shown to disrupt biofilms. The composition may be used for the treatment of any disease, particularly in diseases where intracellular delivery of the particle or the medicament from the particle is advantageous. Treatment may include disease of the urinary tract, including urinary tract infection and other bladder related diseases. The particle may include any medicament for the treatment of disease. This highly cell-penetrative form of medicament could be introduced directly into the urinary tract via catheter or other intravesical conduit. The invention may also find utility in a number of indications which require robust intracellular or biofilm penetration, such as bacterial infections. It has been found that medicaments, such as certain antibiotics, can have improved efficacy when in the polymeric particles compared to the free medicament at the same concentration.

### BRIEF DESCRIPTION OF THE FIGURES

FIG. 1 is an image of the exterior of the spraying chamber, used for synthesising example particles, with some accessory equipment necessary for processing.

FIG. 2 is an image of the interior of the spraying chamber, used for synthesising example particles, with the quadra-axial needle seen in the centre.

FIG. 3 demonstrates aschematic of various example particle formulations (Example Particles 1-5 and Reference Example Particle 6).

FIG. 4 shows an image of the cone-jet during the electrospraying process used for example particle synthesis.

FIG. 5 is a micrograph taken at a) 5×, b) 20× magnification of CapFuran, (Example Particle 1), after resuspension.

FIG. 6 is a scanning electron micrograph of CapFuran (Example Particle 1), on a glass slide.

FIG. 7 is a scanning electron micrograph of CapFuran-FITC an (Example Particle 2), on a glass slide.

FIG. 8 is a scanning electron micrograph of CapFuran-PF127 (Example Particle 3) on a glass slide.

FIG. 9 is a scanning electron micrograph of Cap-Furan-FITC-PF127 (Example Particle 4) on a glass slide.

FIG. 10 demonstrates a field of bladder cells in culture onto which CapFuran-FITC (Example Particle 2) has been added during treatment. The white arrows indicate the glowing green capsules.

FIG. 11 (A) shows FTIR spectra demonstrating CapFuran (Example Particle 1), free nitrofurantoin, S-CapFuran (CapFuran which has been sterilised with 20 kGy gamma radiation) and CapFuran-Placebo (Reference Example Particle 6). (B) shows a Raman spectra of the same. (C) shows a killing assay with E. faecalis with CapFuran (Example Particle 1) and S-CapFuran, presenting the number of colony forming units of E. faecalis present in vials which have been co-cultured the treatments as indicated. By day 2, both CapFuran and S-CapFuran have significantly reduced bacterial populations and by day 3, no living bacteria were present

FIG. 12 (A) shows a dose-dependant uptake of CapFuran-FITC (an example particle composed of PLGA, nitrofurantoin and a FITC fluorescent dye) by cells, with 100% uptake shown at 2.5 and 1.25 mg/ml after a 2-hour treatment time. The majority of cells have taken up cargo from CapFuran-FITC particles, and cells still appear to be morphologically healthy. Below 1.25 mg/ml the numbers of cells displaying bright fluorescence decreases. This indicates that delivery of the particles is cell-specific, rather than by free diffusion. (B) The figure further shows that free FITC at the equivalent concentration to what is harboured within the above capsules behaves differently on dilution, namely becoming progressively less intense in all cells, highlighting the difference between targeted capsule delivery and free diffusion of cargo.

FIG. 13 shows cultured bladder cells that have taken up CapFuran-FITC's (Example Particle 2) green cargo after 2 hr. The bright green appearance of positive cells suggests that the capsule-delivered FITC is high in cells, where the dye is present in both the cytoplasm and the nucleus. This demonstrates the cell penetrating ability of CapFuran-FITC.

FIG. 14 (A) shows the human urothelial organoid model (differentiated and stratified into 3-4 layers thick), after uptake of CapFuran-FITC (Example Particle 2) for two hours. The green dye (bright grey haze) is distributed in all layers demonstrating intracellular delivery of particles (which can also be seen intact on the surface [arrows]). (B) An organoid treated with the equivalent amount of free FITC (14 ug/mL) shows no visible (green) uptake at all, except by sporadic dead cells on the surface (arrows). For both (A) and (B), the left image is a 3D view, the top right image is a top-down “maximum projection” view, and the bottom right image is an orthogonal cross-section. Scale bar is 10 micron in all cases. (Quantification of these image data are presented in FIG. 15)

FIG. 15 (A) shows a Z-axis profile plot of penetration depth versus mean fluorescence intensity derived from the experiment depicted in (A) and (B), measured by pixel analysis of the green FITC channel (black line) compared with red phalloidin, which stains the F-actin cell cortex for reference (grey line). CapFuran-FITC demonstrates robust penetration through multiple cell layers, with fluorescence being more concentrated in the top layer and becoming less concentrated in bottom layers, but being present throughout. In contrast, penetration by free FITC is negligible. (B) shows the corrected total cellular fluorescence (CTCF) statistical analysis of FITC penetration in the experiment shown in (A) and (B), analysed two different ways as described above each graph (log scale) and demonstrating that the enormous difference between particle-directed uptake and free diffusion uptake is highly statistically significant.

FIG. 16 (A) shows treatment of a 3D bladder organoid with CapFuran-FITC-4X (Example Particle 5), a brighter version useful for interrogating depth of penetration. This shows robust penetration of the FITC cargo (bright grey haze) throughout the layers of the organoid (3-4 layers thick; “a” indicates the apical surface boundary and “b” indicates the basal, bottom boundary of the tissue). Scale bars are 10 microns. Arrows indicate capsules on the surface. (B) shows a plot of penetration depth versus mean fluorescence intensity derived from the experiment depicted in (A), measured by pixel analysis of the green FITC channel (black line) vs the red Actin cell cortex channel (grey line), which stains throughout the cell layers, confirming strong intracellular delivery of FITC via CapFuran treatment, through multiple cell layers.

FIG. 17 shows the number of colony forming units of E. faecalis present in vials which have been co-cultured with either nitrofurantoin, CapFuran (Example Particle 1) or CapFuran-Placebo (Reference Example Particle 6). This is the result of six separate experiments which have been normalised and averaged; bars indicate standard error of the mean.

FIG. 18 shows the number of colony forming units of different bacterial strains present in vials which have been co-cultured with either nitrofurantoin, CapFuran (Example Particle 1) or CapFuran-Placebo (Reference Example Particle 6). (A) shows the normal 2.0 mg/mL dose of capsules (200 ug/mL dose of free drug) whereas (B) shows the same assay conducted with lower doses of treatment (1.0 mg/mL for Enterobacter and Staphylococcus, and 1.5 mg/mL for Citrobacter, each paired with the equivalent free drug dose (100 ug/mL and 150 ug/mL respectively).

FIG. 19 (a) shows Cytotoxicity of CapFuran capsules is similar or lower than comparable nitrofurantoin treatment in a human urothelial organoid model. Human urothelial organoids were exposed to two CapFuran concentrations, two equivalent free nitrofurantoin concentrations and control (culture media only) treatment. Cytotoxicity was calculated by measuring LDH release (N=6). Culture media caused no cell damage. CapFuran 2 mg/ml revealed a lower amount of cytotoxicity (p=5.23, SD=0.23) compared to the equivalent free nitrofurantion 200 mg/ml dose (p=6.60, SD=0.49). Bars indicate minimum to maximum values and the mean and standard deviations within the boxes.

FIG. 19 (b) shows mean and 95% CI of bacteria enumerated post lysis of urothelial organoid using the Antibiotic Protection Assay. Mean CFU/ml and 95% CI of bacteria enumerated in each treatment category after lysis of bladder organoid during the assay. Experiment was repeated 3 times (N=3). Bladder organoids treated with Capfuran capsules revealed much lower amounts of intracellular bacteria compared with nitrofurantoin (df=3, F=18.891, P=0.017), indicating a superior efficacy.

FIG. 20 shows confocal laser scanning microscopy images of treated E. faecalis biofilms formed on HBLAK-coated glass chamber slides. The top (A-D) and bottom rows (E-H) show above and side views respectively of 3D images produced for each treated biofilm, with the treatments indicated above (2 hours). Numbers and scale bars are measured in μm. Red colour (CTC staining) is greatly increased in D and H, indicating an increase in bacterial respiration.

FIG. 21 shows the biomass of E. faecalis biofilms grown on porous polycarbonate membranes after the indicated treatments (2 hr). Bars show the DAPI:CTC (bacterial DNA:respiring bacteria) biomass ratio for each treatment condition.

FIG. 22 shows a quantitative representation of CapFuran-FITC (Example Particle 2) distribution within treated biofilms of E. faecalis. Histograms (A) and (C) show the Z position (μm) of the capsules in the HBLAK covered Lab-Tek™ (HCLT) and porous polycarbonate membrane models respectively, whereas the histograms (B) and (D) shows the Z position of the biofilm for each model. Collectively, histograms (A) and (B) show the relative position of the capsules and the biofilm for the HCLT biofilm model. Histograms labelled (C) and (D) represent the same, but for the PPM biofilm model.

FIG. 23 is a confocal laser scanning microscopy image of CapFuran-FITC-treated E. faecalis biofilms formed on HBLAK-covered LabTeks. The bright grey ovals (arrow) are individual bacteria illuminated by FITC, showing that the cargo has been taken up by bacteria within the biofilm.

FIG. 24 shows an embodiment of the device for use in producing the particles described herein, the embodiment comprising a four-needle electrohydrodynamic system.

FIGS. 25A and 25B shows the four-needle electrohydrodynamic system of FIG. 24 in more detail.

FIGS. 26A and 26B shows photographs of the needles used in the electrohydrodynamic system of FIGS. 24 and 25, with FIG. 26A showing the separate needles, and FIG. 26B showing the needles assembled.

FIG. 27A shows enumeration of planktonic E. faecalis bacteria surrounding biofilm after overnight treatment. Box plot shows the effect on bacterial count and therefore killing ability of CapFuran capsules versus nitrofurantoin on the supernatant of E. faecalis biofilm. CapFuran were most effective showing approximately a 1.5 log difference in mean log CFU count after treatment compared with pure nitrofurantoin. CapFuran gave a mean of 2.071 [95% CI, 0.22-3.92] versus 3.60 [95% CI, 6.11-6.85] with nitrofurantoin. (N=3).

FIG. 27B shows B) CapFuran capsules are more effective against solid biofilms than nitrofurantoin. A box plot of enumerated bacteria (log CFU/ml) after subsequent mechanical disruption of biofilms that were treated overnight with either CapFuran capsules, blank capsules, media solution or pure nitrofurantoin. CapFuran capsules revealed a 1.6 log difference in mean log CFU counts compared with nitrofurantoin. Capsules mean 4.839 (95% CI 3.515 to 6.162) compared to nitrofurantoin mean 6.479 [95% CI, 6.11-6.85]. (N=3)

FIG. 28 shows still images from a timelapse videomicroscopy series in which CapFuran-FITC was added to human HBLAK bladder cells growing in culture and filmed over a period of time. As shown, the capsules (indicated with black arrows in the first frame) dock within minutes and delivery to the cells occurs without the capsule being taken up; instead, the cargo (bright white haze) is pumped directly into the cells starting between 19 and 22 minutes post treatment and is completed to all cells between 25 and 29 minutes.

### DETAILED DESCRIPTION

The present invention provides the first to the fifth aspects defined above. Also described herein are optional and preferred features of the aspects. Any optional or preferred feature is applicable to any aspect unless specifically stated otherwise, and may be combined with any other optional or preferred feature.

The method of any of the aspects may involve the intracellular delivery of the particle comprising a biodegradable and hydrolysable polymer and the medicament, or the medicament from the particle (with or without the hydrolysable polymer). Intracellular delivery indicates that the particle or medicament is delivered to the interior of a cell of a human or other mammal; it does not indicate the particle or medicament is delivered to the interior of a bacterial cell. The cell may be a diseased human or mammalian cell, optionally an infected cell human or mammalian cell, e.g. infected with bacteria, which may be as described herein.

The particles comprise a biodegradable and hydrolysable polymer. The polymer may be a homopolymer or a co-polymer of two or more different types of monomer. The biodegradable and hydrolysable particle is degraded into oligomers and/or monomers by hydrolysis, which, in the present context means the links between at least some of the monomer units are hydrolysed, resulting in a chain shortening of the monomer. This may result in the release of the medicament within a cell. The particle may be additionally degraded by enzymes. The polymer preferably has linkages between monomer units selected from ester linkages, anhydride linkages and amide linkages. The term ester linkage is defined as any linkage which has the chemical formula —O—C(═O)—. Examples of polymers with ester linkages include, but are not limited to: Poly(lactic-co-glycolic acid) (PLGA), Polycaprolactone (PCL), Polyglycolic acid (PGA), Polylactic acid (PLA) or any polyhydroxyalkanoate (PHA), including polyhydroxybutyrate (PHB); the polymer may be or comprise any of these types of polymers with ester linkages. PLGA may have any ratio of glycolic acid and lactic acid monomers. In one example, the molar ratio of glycolic acid monomer:lactic acid monomer is from 10:90 to 90:10, optionally from 20:80 to 80:20, optionally from 30:70 to 70:30, optionally from 40:60 to 60:40, optionally about 50:50. Any co-polymers of PLGA, PCL, PHA, PGA, PLA or a PHA may also be used.

The polymer may have anhydride linkages between monomer units for efficient hydrolysis. The term anhydride linkage is defined as any linkage which has the chemical formula —C(═O)—O—C(═O)—. Examples of hydrolysable polymers with anhydride linkages include but are not limited to: poly[bis(p-carboxyphenoxy)methane (PCPB), Poly[1,6-bis(p-carboxyphenoxy)hexane] (Poly CPH), and any Poly(glycerol sebacate) (PGS). The polymer may be or comprise a protein formed of amino acid monomers, a nucleic acid formed of nucleotide monomers, or a polysaccharide formed from carbohydrate monomers.

The polymer may allow for controlled drug delivery of the medicament, for example an initial burst release of the medicament and/or gradual released of the medicament due to polymer degradation. Examples of particularly suitable polymers include, but are not limited to, PLGA, PGA, PLA, PCL, PHA and PHB. Such polymers have been found to have short half lives, in vivo, i.e. much shorter than, for example, a polymer such as PMSQ. It has also been found that the particles described herein, which include these polymers and having the sizes mentioned herein, have a high propensity to penetrate cells, e.g. cells of the urinary tract, such as cells of the bladder. Such polymers have also been found to be soluble in low toxicity solvents such as acetone. The polymer may be FDA and/or MHRA and/or EMA approved for medical use. The polymer may be hydrolysed to monomers that are common metabolites in higher organisms, in other words, metabolites that can be metabolised by the Krebs cycle without need for excretion by the liver or the kidneys. This is advantageous to minimise systemic toxicity. Examples of these polymers include, but are not limited to: PLGA which hydrolyses to glycolic acid and lactic acid, PGA which hydrolyses to glycolic acid, PLA which hydrolyses to lactic acid and PHB which hydrolyses to 3-hydroxybutyric acid.

According to both first and second aspects, the medicament may be selected from pharmaceutical and/or cosmetic active agents, which may be selected from growth factors; growth factor receptors; transcriptional activators; translational promoters; antiproliferative agents; growth hormones; anti-rejection drugs; anti-thrombotic agents; anti-coagulants; stem cell or gene therapies; antioxidants; free radical scavengers; nutrients; co-enzymes; ligands; cell adhesion peptides; peptides; proteins; nucleic acids; DNA; RNA; sugars; saccharides; nutrients; hormones; antibodies; immunomodulating agents; growth factor inhibitors; growth factor receptor antagonists; transcriptional repressors; translational repressors; replication inhibitors; inhibitory antibodies; cytotoxin; hormonal agonists; hormonal antagonists; inhibitors of hormone biosynthesis and processing; antigestagens; antiandrogens; antiinflammatory agents; non-steroidal antiinflammatory agents (NSAIDs); analgesics; COX-I and II inhibitors; antibiotics, antimicrobial agents; antiviral agents; antifungal agents; anti-proliferative agents; antineoplastic/antiproliferative/anti-mitotic agents; anesthetic, analgesic or pain-killing agents; antipyretic agents, prostaglandin inhibitors; platelet inhibitors; DNA de-methylating agents; cholesterol-lowering agents; vasodilating agents; endogenous vasoactive interference agents; angiogenic substances; cardiac failure active ingredients; polysaccharides; sugars; targeting toxin agents; aptamers; quantum dots; nano-materials; nano-crystals; and combinations thereof. Any further suitable active agents or medicaments could be used which are well known to those of skill in the art and include, by way of non-limiting example, those disclosed in the Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and U.S. Pat. Nos. 6,589,562, 6,825,161, 6,063,365, and 6,491, 902, all to Shefer et al, each incorporated herein by reference.

In one embodiment, the medicament may be or comprise an antibiotic. The antibiotic may be for treating gram-positive or gram-negative bacteria. The antibiotic may be an antibiotic used for the treatment of a urinary tract infection (UTI). This may include but are not limited to ampicillin, ceftriaxone, cephalexin, ciprofloxacin, gentamicin, fosfomycin, levofloxacin, trimethoprim/sulfamethoxazole, and nitrofurans including nitrofurantoin. In an example, the antibiotic used is nitrofurantoin. The antibiotic may be a cephalosporin, e.g. s cephalosporins selected from cefpodoxime, cefdinir and cefaclor.

In another embodiment of both the first and second aspect, the particle contains an antimuscarinic or anticholigenic as an active agent or medicament. This may include but is not limited to onabotulinumtoxinA (tradename: Botox), oxybutynin, solifenacin, tolterodine, fesoterodine, trospium, oxybutynin chloride and darifencin. In another embodiment of the first and second aspect, the particle contains a chemotherapeutic or immunotherapeutic agent.

In an embodiment of both the first and second aspect, the particles may additionally comprise a diagnostic agent. This agent should be suitable for use in a technique selected from, but not limited to, diagnostic medical imaging procedures (for example, radiographic imaging (x-ray), fluorescence spectroscopy, Forster/fluorescent resonance energy-transfer (FRET), computed tomography (CT scan), magnetic resonance imaging (MRI), positron emission tomography (PET), other nuclear imaging, and the like. The diagnostic agent may be an agent for use in diagnostic imaging, for example a contrast agent, such as barium sulfate for use with MRI. An example of a fluorescent dye is, but is not limited to, fluorescein isothiocyanate (FITC). Fluorescent agents may be used to trace medicament delivery and/or cell penetration efficiency.

The wt:wt ratio of the medicament (e.g. antibiotic):polymer in the particle(s) may be 1:100 to 1:2, optionally 1:100 to 1:5, optionally 1:50 to 1:5, optionally 1:20 to 1:5, optionally about 1:10 or about 1:11. The medicament (e.g. antibiotic) may be present in the particle(s) in an amount of 0.01 wt % to 50 wt % of the particle(s), optionally an amount of 0.01 wt % to 40 wt %, optionally 0.01 wt % to 30 wt %, optionally 0.1 wt % to 30 wt %, optionally 0.1 wt % to 20 wt %, optionally 0.1 wt % to 15 wt %, optionally 1 wt % to 15 wt %, optionally 5 wt % to 15 wt %, optionally 8 wt % to 12 wt %.

The particles may also include a surfactant. The surfactant may be incorporated within the particle or be present on the particle surface. The surfactant may be either ionic or non-ionic. An ionic surfactant may be incorporated within the particle to improve their suspension characteristics. The ionic surfactant may be selected from an anionic surfactant and a cationic surfactant. An example of a non-ionic surfactant includes, but is not limited to, Pluronic F127.

In an embodiment, the particles may comprise a biocompatible surfactant, preferably a non-ionic surfactant. The surfactant may be any copolymer formed from both a hydrophobic and hydrophilic monomers. Preferably, the surfactant may be any polymer or copolymer which comprises a C1-010 polyoxyalkylene, optionally a C2-C4 polyoxyalkylene. Polyoxyalkylene may be referred to as polyols or polyalkylene glycols (e.g. poly(ethylene glycol) or poly(propylene glycol) in the art. Preferably, the surfactant is a copolymer of a C2-C4 hydrophilic polyoxyalkylene and a C2-C4 hydrophobic polyoxyalkylene. The surfactant may be a triblock polymer comprised of a C2-C4 hydrophobic polyoxyalkylene central portion and two hydrophilic C2-C4 polyoxyalkylene flanking regions. In an embodiment, the surfactant may be a poloxamer block co-polymer comprising two polyoxyethylene terminal portions and a polyoxypropylene central portion with the general formula (I) HO[CH₂CH₂O]ₐ₁ [CH(CH₃)CH₂O]_{b}[CH₂CH₂O]ₐ₂H, wherein a1 and a2 are each independently=2-130 and b=10-100, optionally wherein a1 and a2 are each independently=70-130 and b=25-67, optionally wherein the polyoxyethylene content is greater than 40% by wt, and preferably 70% by wt calculated using number average molecular weights.

In a preferred embodiment, the surfactant has the formula (I) wherein a1 and a2 are each independently=95-105 and b=54-60 and optionally the polyoxyethylene content is greater than 70% by wt. Any combination of surfactants or poloxamers may be used.

The surfactant may have a number average molecular weight less than 25000 Da, preferably less than 20000 Da, preferably less than 18000 Da, preferably less than 15000 Da, preferably less than 13000 Da. The surfactant may have a number average molecular weight of at least 3000 Da, preferably at least 5000 Da, preferably at least 7500 Da, preferably at least 10000 Da or preferably at least 12000 Da. In an example, the surfactant has a number average molecular weight between 12,300 and 12,700 Da.

The surfactant may have a HLB (hydrophilic lipophilic balance) of at least 10, preferably at least 12, preferably at least 16, preferably at least 18, preferably at least 20 or preferably at least 21. In an example, the surfactant has a HLB between 18-23, more specifically a HLB of 22.

The surfactant may be selected from Poloxamer 407 (Tradename: Pluronic F-127/PF-127, Synperonic PE/F 127), Poloxamer 181 (Tradename: Pluronic L61, Superonic PE/L 61), Poloxamer 123 (Pluronic-L 44), Poloxamer 237 (Tradename: Pluronic F 87) or Poloxamer 338 (Tradename: Pluronic F108) wherein the first two digits×100 give the number average molecular mass of the polyoxyethylene and the last digit×10 gives the % content of polyoxyethylene by wt. In an example, the surfactant used is Poloxamer 407.

The wt:wt ratio of the surfactant:polymer in the particle(s) may be 1:100 to 1:2, optionally 1:100 to 1:5, optionally 1:50 to 1:5, optionally 1:20 to 1:5, optionally about 1:10 or about 1:11. The surfactant may be present in the particle(s) in an amount of 0.01 wt % to 50 wt % of the particle(s), optionally an amount of 1 wt % to 50 wt %, optionally 1 wt % to 40 wt %, optionally 1 wt % to 30 wt %, optionally 1 wt % to 20 wt %, optionally 3 wt % to 17 wt %, optionally 5 wt % to 15 wt %, optionally 8 wt % to 12 wt %.

In an example, the addition of a surfactant is found to improve the suspension characteristics of the particle. It is thought that the surfactant may also improve the ability of particles or medicament from the particles, to penetrate cells, facilitating their intracellular delivery in the bladder.

According to both first and second aspects, the particles may have any suitable shape or form. The particles may have a mainly spherical morphology. The particles may be capsules. The capsules may have a solid-shell and an inner core. The particles may have a layer structure with any number of layers. Inner layers may comprise a solid, liquid or gas. The medicament may be present in at least one or all of the particle layers. The layers may all comprise the same polymer, or different layers may be comprised of different polymers.

According to both first and second aspects, the particles have a particle size, e.g. a number average particle size, in a range of 1 μm-30 μm. The particles may have an average particle size, e.g. a number average particle size, less than 30 μm, optionally less than 25 μm, optionally less than 20 μm, optionally less than 15 μm, optionally less than 10 μm, optionally less than 5 μm, optionally less than 4 μm, optionally less than 3 μm. This range of particle size is thought to aid cell-penetration. Particle size may be measured by any suitable means such as scanning electron microscopy (SEM). SEM measures the mean diameter across the particles. Optionally, this relates to the mean diameter of the particle at its largest point.

The composition may comprise a plurality of the particles comprising the biodegradable and hydrolysable polymer. The composition may comprise a plurality of the particles comprising the biodegradable and hydrolysable polymer, wherein at least 90%, by number, of the particles comprising the biodegradable and hydrolysable polymer have a diameter, as measured using a scanning electron microscope, of 10 μm or less.

The composition may comprise a plurality of the particles comprising the biodegradable and hydrolysable polymer, wherein at least 90%, by number, of the particles comprising the biodegradable and hydrolysable polymer have a diameter, as measured using a scanning electron microscope, of from 1 to 10 μm.

The dimension or diameter of a particle may be determined by placing a sample of the composition on a surface, e.g. of a slide, and measuring the largest dimension across a particle. The percent, by number, of the particles having a diameter within a certain range may be measured by taking a sample of the composition, and, within that sample, counting the total number of particles, and measuring the diameter of each particle within the sample; the percent of particles within a certain size range is: (the number of particles having a diameter within that size range in the sample/total number of particles within a sample)×100. It is considered that keeping the size of the particles within the ranges mentioned above increases the propensity of the particles to be delivered intracellularly. While nanoparticles have been found effective in the prior art for delivering certain active agents intracellularly, larger particles of, say, 1 μm, have not, before the present invention, been found to be particularly effective. However, the present inventors found that the particles described here are surprisingly effective in being delivered intracellularly.

In an embodiment of any of the aspects, e.g. the first and second aspect, the particles are synthesised by electrohydrodynamic processing. The polymer may be selected so that it has suitable chemical properties for electrohydrodynamic processing. This may be carried out using a cone-jet regime which is a stable form of electrospraying which leads to particles with a small size distribution. In one particular embodiment, the electrohydrodynamic apparatus uses a quadra-axial needle.

According to both the first and second aspects, the particles may be delivered by any means for treatment of a disease or a method of treatment. They may be delivered in an embodiment, the particles and medicament are delivered locally or systemically (e.g. orally or intravenously). Preferably, the particles are delivered locally to an area of a host that is affected with the disease. The particles may, for example, be delivered locally to the urinary tract. The particles may be delivered via a synthetic conduit to the urinary tract. The synthetic conduit may be or comprise a tube, which may be a polymeric or metallic tube. The synthetic conduit may comprise a tube comprising a material selected from silicone rubber, nylon, polyurethane, polyethylene terephthalate (PET), latex, and thermoplastic elastomers. The synthetic conduit may be or comprise a catheter or a cannula. For local delivery to the urinary tract, the synthetic conduit may be inserted into the urethra (e.g. using an intermittent or an indwelling catheter) or via an incision in the abdomen (e.g. using a suprapubic catheter). Alternatively, the particles may be delivered by injection, transdermally or via an implanted medical device.

In an embodiment of the first and second aspect, the particles can be used as treatment for diseases in the urinary tract. The diseases may be in any part of the urinary tract, e.g. any part selected from the kidneys, ureters, urinary bladder, prostate and urethra. This may include, but is not limited to, cancer of the urinary tract. This may include bladder cancer, kidney cancer or prostrate cancer. The medicament may be a chemotherapeutic agent or an immunotherapeutic agent. In another embodiment of the first and second aspect, the particles can be used as treatment for diseases of the bladder. This may include, but is not limited to, UTI, bladder cancer and over-active bladder. In the case of UTI treatment, the medicament may be an antibiotic. In the case of over-active bladder, the medicament may be an antimuscarinic. In the case of bladder cancer, the medicament may be a chemotherapeutic agent or an immunotherapeutic agent.

According to the second aspect and in an embodiment of the first aspect, the particles are used for intracellular delivery of the medicament. In an example, the particles enable more effective intracellular delivery of the medicament compared to free diffusion. This enables a higher intracellular concentration of medicament. In addition, the example particle is shown to penetrate multiple layers of cells. This is advantageous since in traditional drug administration methods the medicament is only delivered to the superficial layer. The particles may be used for any method or treatment where intracellular delivery and/or penetration of multiple cell layers is desirable. This may be particularly relevant in the treatment of UTI since in addition to colonization of the apical umbrella cells, the infected bladder also suffers from deeper quiescent intracellular reservoirs further down the bladder wall; this makes total eradication difficult using traditional treatment methods.

The composition may comprise the particles in a liquid medium, preferably a biocompatible liquid medium, e.g. an aqueous liquid medium, e.g. a saline solution, e.g. a saline solution for administration to a human. The composition may comprise a suitable amount of particles in a liquid medium that allows for a suitable effective, but biologically safe, amount of the medicament is delivered. For example, for a medicament, e.g. an antibiotic (e.g. nitrofurantoin), the composition may comprise the particles, such that the composition comprises from 1 μg/ml to 500 μg/ml of the medicament in the composition, in some examples from 10 μg/ml to 500 μg/ml, optionally from 50 μg/ml to 500 μg/ml, optionally from 50 μg/ml to 400 μg/ml, optionally from 50 μg/ml to 300 μg/ml, optionally from 100 μg/ml to 300 μg/ml, optionally from 150 μg/ml to 250 μg/ml in the composition.

In an embodiment of the first and second aspect, the particles are used to treat bacterial infection. The particles may be used to treat bacterial infections including but not limited to: E. faecalis, E. coli, Enterobacter, Klebsiella, Staphylococcus, Citrobacter, Streptococcus pneumoniae, Chlamydophila, Legionella, Salmonella, Neisseria, Brucella, Mycobacterium, Nocardia, Listeria, Francisella, Yersinia, or Coxiella. Example particles demonstrate efficacy against E. faecalis, E. coli, Staphylococcus, Enterobacter and Citrobacter bacteria.

In an embodiment of the first and second aspect, the particles are used in the treatment of biofilms. The biofilms may be formed from bacterial infection. Example particles are shown to be efficacious against biofilms. This is an improvement over medicaments administered by free diffusion since biofilms are naturally resistant to free antibiotics due to a variety of mechanisms. This includes both a physical barrier to drug diffusion, as well as lack of active growth and division.

In an embodiment, there is provided a composition comprising a particle comprising a biodegradable and hydrolysable polymer, wherein a medicament is dispersed in the polymer, wherein the particle has a dimension of from 1 μm to 30 μm, wherein the composition is for use in a method for treating bacteria and optionally the method involves the intracellular delivery of the particle or the medicament from the particle (which may be with or without the hydrolysable polymer). The bacteria may be intracellular bacteria, e.g. within a cell of a host such as a human or animal. Optionally, the composition is for use in the treatment of a biofilm and the treatment of a biofilm preferably results in the killing of at least some bacteria in the biofilm. Optionally, the biofilm contains bacteria selected from E. faecalis, faecalis, E. coli, Enterobacter, Klebsiella, Staphylococcus, Citrobacter, Streptococcus pneumoniae, Chlamydophila, Legionella, Salmonella, Neisseria, Brucella, Mycobacterium, Nocardia, Listeria, Francisella, Yersinia, or Coxiella. Example particles demonstrate efficacy against E. faecalis, E. coli, Staphylococcus, Enterobacter and Citrobacter bacteria. The method of treating bacteria may involve the contacting of the composition with the bacteria, e.g. in a biofilm, e.g. for a time period sufficient such that at least some of the bacteria are killed by the composition. The period may be at least 1 minute, optionally at least 5 minutes, optionally at least 15 minutes, optionally at least 30 minutes, optionally at least an hour. The period may be from 1 minute to 72 hours, optionally 1 minute to 48 hours, optionally 5 minutes to 48 hours, optionally 30 minutes to 48 hours, optionally 30 minutes to 24 hours. The biofilm may be in or on an animal body, e.g. human body, and may be associated with a bacterial infection, which may be as described herein. The biofilm may be on or in a host, e.g. a human or animal, or may be on or in an inanimate object, e.g. on the surface of an inanimate object.

Also provided is a process for producing particles comprising a biodegradable and hydrolysable polymer, the process comprising:
(i) providing an electrohydrodynamic device comprising at least two concentrically arranged, spaced apart hollow needles, the needles together defining a core channel, and an outer concentrically disposed tubular channel; and a means for applying a voltage to the needles
(ii) passing fluid mediums through the hollow core channel, and the outer concentrically disposed tubular channel, wherein at least one of the fluid mediums in one of the channels has therein a biodegradable and hydrolysable polymer and a medicament, optionally for the treatment of a disease of the urinary tract,
(iii) applying a voltage to the needles, such that, on leaving the needles, the particles comprising the biodegradable and hydrolysable polymer are formed, wherein the medicament, optionally for the treatment of a disease of the urinary tract, is dispersed in the polymer, and at least some of the particles have a dimension of from 1 μm to 30 μm.

Preferably, the device comprises at least four concentrically arranged, spaced apart hollow needles, the needles together defining a core channel, at least two intermediate channels and an outer concentrically disposed tubular channel, and fluid mediums are passed down the core channel, the at least two intermediate channels and an outer concentrically disposed tubular channel, and at least one of the fluid mediums in one of the channels comprises a biodegradable and hydrolysable polymer and a medicament for the treatment of a disease of the urinary tract.

Also described herein is an electrohydrodynamic device for producing the particles,
the device comprising
   at least two concentrically arranged, spaced apart hollow needles, the needles together defining a core channel, and an outer concentrically disposed tubular channel; and
   a means for applying a voltage to the needles.

Also described herein is an electrohydrodynamic device for producing the particles,
the device comprising
   at least three concentrically arranged, spaced apart hollow needles, the needles together defining a core channel, at least one intermediate concentrically disposed tubular channels, and an outer concentrically disposed tubular channel; and
   a means for applying a voltage to the needles.

Also described herein is an electrohydrodynamic device for producing the particles,
the device comprising
   at least four concentrically arranged, spaced apart hollow needles, the needles together defining a core channel, at least two intermediate concentrically disposed tubular channels, and an outer concentrically disposed tubular channel; and
   a means for applying a voltage to the needles.

The needles are able to be charged by applying a voltage to the needles. The needles are preferably made from an electrically conducting material, preferably a metal. The metal may be selected from, for example, an elemental metal or a metal alloy. The metal may, for example, comprise steel.

The innermost needle in the device, which defines the hollow core, may have an inner diameter of at least 0.01 mm, preferably at least 0.1 mm. The innermost needle in the device, which defines the hollow core, may have an inner diameter of from 0.01 mm to 2 mm, optionally from 0.05 to 1.5 mm, optionally from 0.15 to 1.0 mm, optionally from 0.15 to 0.40 mm, optionally about 0.3 mm.

The space between the outer surface of a needle and the inner surface of the outwardly disposed adjacent needle may be from 0.01 mm to 1.5 mm, preferably 0.1 mm to 1 mm, optionally 0.2 to 0.9 mm, optionally 0.3 to 0.7 mm, optionally about 0.5 mm. Optionally the space between the outer surface of each needle (except the outer needle) and the inner surface of the outwardly disposed adjacent needle may be from 0.01 mm to 1.5 mm, preferably 0.1 mm to 1 mm, optionally 0.2 to 0.9 mm, optionally 0.3 to 0.7 mm, optionally about 0.5 mm.

In an embodiment, the device comprises an inner needle, which defines the hollow core, which has an inner diameter of from 0.15 to 1.0 mm, optionally from 0.15 to 0.45 mm, optionally about 0.3 mm, at least three needles disposed outwardly in a concentric manner from the innermost needle, wherein the space between the outer surface of each needle (except the outer needle) and the inner surface of the outwardly disposed adjacent needle is from 0.01 mm to 1.5 mm, preferably 0.1 mm to 1 mm, optionally 0.2 to 0.9 mm, optionally 0.3 to 0.7 mm, optionally about 0.5 mm.

The means for applying a voltage to the needles may apply any suitable voltage. The voltage may be from 1 kV to 50 kV, preferably 3 kV to 30 kV, more preferably 15 kV to 25 kV, optionally about 17 kV. The means for supplying a voltage may apply a dc voltage or an ac voltage, optionally a dc voltage. A ground electrode may be present, which may be at or near the collection means. The ground electrode may be placed at any suitable distance from the needles, for example a distance of from 1 mm to 1 m, optionally 1 mm to 50 cm, optionally 1 mm to 30 cm, optionally 10 cm to 30 cm.

The device optionally further comprises means for supplying a fluid to each channel. The means for supplying a fluid to each channel preferably can supply a fluid medium selected from the first and second fluid medium. Preferably, at least one of the intermediate concentrically disposed channels is in fluid connection with a means for supplying the first fluid medium; and optionally the remaining channels are in fluid connection with a fluid medium selected from the first fluid medium and the second fluid medium. The means for supplying a fluid to each channel preferably comprises a syringe pump or pressurised vessel. Preferably a syringe pump or pressurised vessel is in fluid connection with one end of each channel. Each means for supplying a fluid can preferably supply a fluid medium at a rate of from 1 μl/min to 2000 μl/min, optionally from 5 to 200 μl/min, optionally from 5 to 50 μl/min.

Optionally, the device further comprises a collection means for collecting the fluid mediums exiting the needles and/or the layered body or bodies formed therefrom. The collection means is preferably earthed. The collection means may be a receptacle.

The device may further comprise a means for observing the fluid mediums exiting the needles and/or the layered body or bodies formed therefrom. The means for observing may comprise a camera, optionally connected to a recording means. The camera may optionally be connected to a visual display means, so that the fluid mediums exiting the needles and/or the layered body or bodies formed therefrom exiting the needles can be observed.

Each of the fluid mediums passed through the channels may comprise a liquid comprising a non-volatile component, e.g. the biodegradeable and hydrolysable polymer. The fluid medium having therein the biodegradable and hydrolysable polymer and the medicament may be termed a first fluid medium; the biodegradable and hydrolysable polymer may be dissolved or suspended in the first fluid medium. The liquid of the fluid medium may also be a volatile or a non-volatile component. The liquid may have a boiling point of at least 40° C., optionally at least 50° C., optionally at least 100° C., optionally at least 150° C., optionally at least 200° C., optionally at least 250° C. The liquid may have a boiling point of from 40° C., to 100° C., optionally from 40° C. to 80° C., optionally from 40° C. to 70° C., optionally from 50° C. to 60° C. All boiling and melting points given herein, unless otherwise stated, are measured at standard pressure (101.325 kPa). The liquid may comprise an organic solvent. The organic solvent may comprise a non-polar solvent and/or a polar solvent. The organic solvent may comprise an aprotic solvent and/or a protic solvent. Non-polar solvents include, but are not limited to, pentane, cyclopentane, hexane, benzene, toluene, 1,4-dioxane, chloroform, and diethylether. The solvent may comprise a polar aprotic solvent, optionally selected from dichloromethane, tetrahydrofuran, ethylacetate, acetone, dimethylformamide, acetonitrile, dimethyl sulphoxide. The solvent may comprise a polar protic solvent, optionally selected from formic acid, n-butanol, isopropanol, n-propanol, ethanol, methanol and acetic acid. The organic solvent may comprise a hydrocarbon. The hydrocarbon may comprise an aromatic or an aliphatic hydrocarbon. The hydrocarbons may be selected from, but are not limited to, pentane, cyclopentane, hexane, cyclohexane and benzene.

The polymer may be dissolved in the first fluid medium in an amount of at least 1 mg of polymer per ml of liquid of the first fluid medium (i.e. 1 mg/ml), optionally at least 5 mg/ml, optionally at least 10 mg/ml, optionally at least 15 mg/ml, optionally at least 20 mg/ml, optionally at least 25 mg/ml. The polymer may be dissolved in the first fluid medium in an amount of from 1 mg of polymer to 200 mg per ml of liquid of the first fluid medium (i.e. 1 to 200 mg/ml), optionally 1 mg/ml to 100 mg/ml, optionally 5 mg/ml to 100 mg/ml, optionally 10 mg/ml to 80 mg/ml, optionally 10 mg/ml to 50 mg/ml, optionally 20 mg/ml to 40 mg/ml, optionally 20 mg/ml to 40 mg/ml, optionally about 30 mg/ml.

The medicament may be present in the first fluid medium in a suitable amount that will represent the weight percent of the medicament in the polymer of the particle. The wt:wt ratio of the medicament:polymer (in the first fluid medium and in the particles) may be 1:100 to 1:2, optionally 1:100 to 1:5, optionally 1:50 to 1:5, optionally 1:20 to 1:5.

Typically, the particles produced by the method contain little, if any of the first fluid medium, since this will typically evaporate or otherwise disassociate from the particle during the method, e.g. as the particles exit the needles or shortly afterwards.

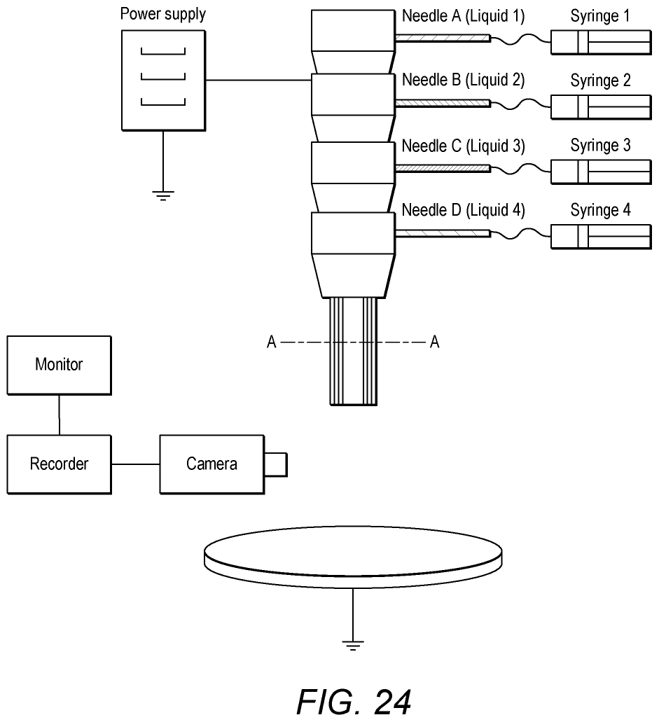

FIGS. 24, 25 and 26 shows an embodiment of the electrohydrodynamic device of the present invention. The device comprises four concentrically arranged, spaced apart hollow needles A, B, C and D, the needles together defining a core channel W, two intermediate concentrically disposed tubular channels X and Y, and an outer concentrically disposed tubular channel Z; and a means for applying a voltage to the needles.

As shown in FIG. 24, a syringe pump or other supply device is fluidly connected to one end of each channel. The inner surface of the innermost needle A defines the core channel W. The core channel W is fluidly connected to syringe 1, which may be via any suitable conduit such as a tube, preferably a tube comprising silicone.

The outwardly disposed adjacent needle to the core needle is intermediate needle B. An intermediate concentrically disposed tubular channel X is defined by the outer surface of innermost needle A and inner surface of needle B. Tubular channel X is fluidly connected to syringe 2, which may be via any suitable conduit such as a tube, preferably a tube comprising silicone.

The outwardly disposed adjacent needle to intermediate needle B is intermediate needle C. An intermediate concentrically disposed tubular channel Y is defined by the outer surface of needle B and inner surface of needle C. Tubular channel Y is fluidly connected to syringe 3, which may be via any suitable conduit such as a tube, preferably a tube comprising silicone.

The outwardly disposed adjacent needle to intermediate needle C is outermost needle D. An outer concentrically disposed tubular channel Z is defined by the outer surface of innermost needle C and inner surface of needle D. Tubular channel Z is fluidly connected to syringe 4, which may be via any suitable conduit such as a tube, preferably a tube comprising silicone.

As shown in FIGS. 24, 25 and 26, all needles have a free end through which the fluid mediums being passed through the needles can exit. The fluid mediums exiting the needles will together be termed a fluid composition from hereon. At the end of the needles distal to the free end, each of needles A, B and C is in flush connection with the adjacent outwardly disposed needle.

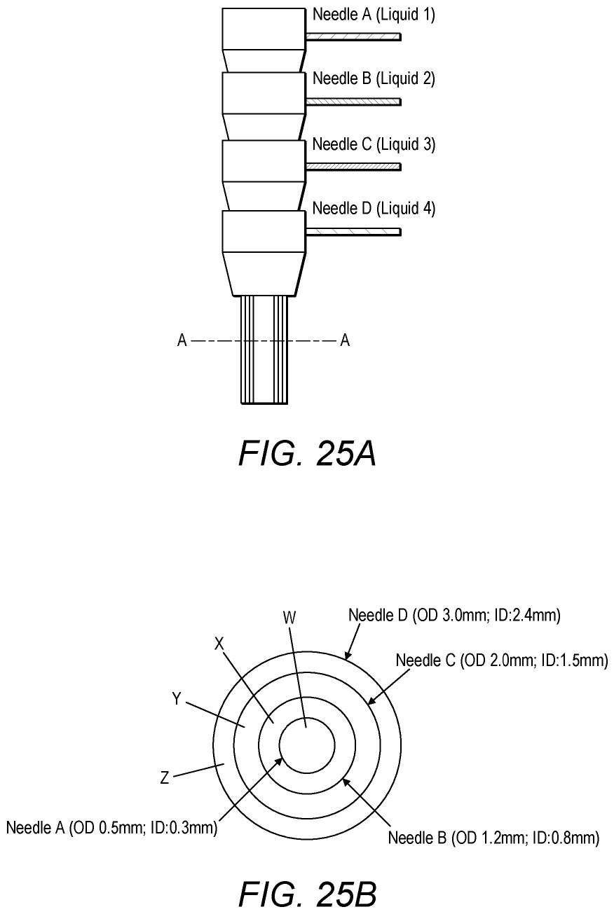

FIG. 25B shows a cross-sectional view of the needles and the channels they define along the line A-A of FIG. 25A. FIG. 25B shows the dimensions of a particular embodiment of the needles, as used in the Examples below. These dimensions can be varied and may be otherwise as described herein, depending on the desired size of the particles or threads that the skilled person wishes to produce with the device. In FIG. 25B, ID and OD represent, respectively, inner diameter and outer diameter.

In use, the needles may be orientated so that the axis of the needles is substantially vertical.

As shown in FIG. 24, a collection means is provided for collecting the layered body or bodies formed from the fluid composition after exiting the needles. The collection means is earthed. The collection means is disposed below the needles. The collection means may be in any suitable form, for example a plate or a receptacle. It may, for example be a metallic, e.g. a steel, plate or receptacle.

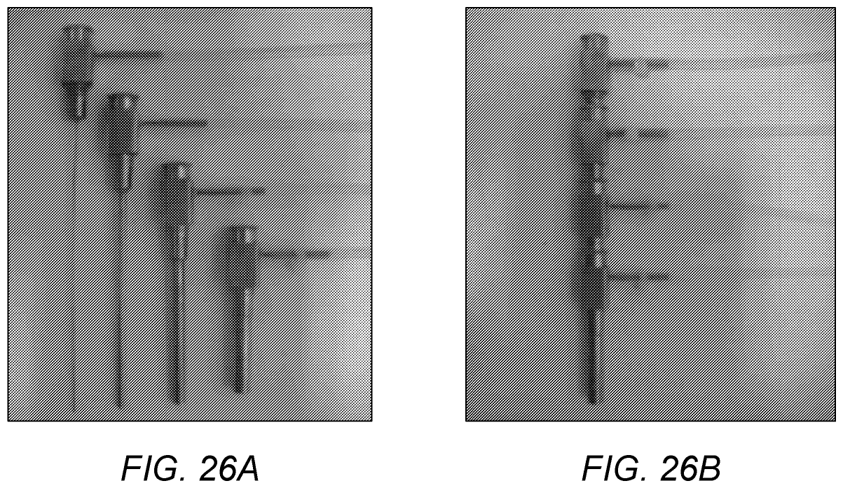

FIGS. 26A and 26B show photographs of the needles used in the electrohydrodynamic system of FIGS. 24 and 25, with FIG. 26A showing the separate needles, and FIG. 26B showing the needles assembled. In FIG. 26A, the left hand needle is needle A, with needles B, C and D shown in order to the right.

In use, syringe 1 can provide a liquid 1 to channel W, preferably a first or second fluid medium as described herein. In use, syringe 2 can provide a liquid 2 to channel X, which may be a first or second fluid medium as described herein. In use, syringe 3 can provide a liquid 3 to channel Y, which may be a first or second fluid medium as described herein. In use, syringe 4 can provide a liquid 4 to channel Z, which may be a first fluid medium as described herein. Preferably at least two, optionally at least three, optionally at least four of the channels contain a first fluid medium as described here, the first fluid medium containing the polymer, the medicament, and optionally the surfactant, as described herein. Each of the syringes preferably supplies a fluid medium, e.g. the first or second fluid medium as described herein, at a suitable rate, e.g. a rate of from 1 μl/min to 2000 μl/min, to the channel to which it is fluidly connected. The rate of supply of the fluid medium for each channel may be the same as or different to one or more of the other channels, e.g. the adjacent channel disposed outwardly or inwardly.

Optionally, a second fluid medium comprising a volatile liquid may be passed down a channel other than the channel(s) down which the first fluid medium is passed. Optionally, the second fluid medium may be passed down a channel disposed concentrically inwardly of the channel or channels down which the first fluid medium is passed; for example the second fluid medium may be passed down the core channel (i.e. the centre-most channel) and the first fluid medium passed down one or more channels disposed concentrically outwardly from the core channel. Optionally, if a first fluid medium, as described herein, is passed through two adjacent channels, preferably, the first fluid mediums in one of these channels is immiscible with the first fluid medium in the adjacent channel. The first fluid mediums should be sufficiently immiscible such that two distinct phases form in the layered bodies. A person skilled in the art of electrohydrodynamic techniques could select appropriate first fluid mediums.

The second fluid medium comprises or consists of a volatile liquid. In an embodiment, the volatile liquid is a liquid that has a boiling point not higher than 50° C. above the temperature of the environment into which the fluid mediums pass into when exiting the channels, optionally not higher than 40° C. above the temperature of the environment into which the fluid mediums pass into when exiting the channels, optionally not higher than 35° C. above the temperature of the environment into which the fluid mediums pass into when exiting the channels, optionally not higher than 30° C. above the temperature of the environment into which the fluid mediums pass into when exiting the channels. For example, if the temperature of the environment into which the fluid mediums pass into when exiting the channels is 25° C., preferably, the volatile liquid has a boiling point of 75° C. or less. The volatile liquid may have a boiling point of less than 100° C., optionally less than 80° C., optionally less than 70° C., optionally less than 60° C., optionally less than 50° C.

The temperature of the environment into which the fluid mediums pass into when exiting the channels may be any suitable temperature. The process may be carried out such that the temperature of the environment into which the fluid mediums pass into when exiting the channels is at or above the boiling point of the liquid of the second fluid medium. The temperature of the environment into which the fluid mediums pass into when exiting the channels may be above 15° C., optionally above 20° C., optionally above 25° C. The temperature of the environment into which the fluid mediums pass into when exiting the channels may be less than 150° C., optionally less than 100° C., optionally less than 80° C., optionally less than 60° C., optionally less than 40° C. The temperature of the environment into which the fluid mediums pass into when exiting the channels may be from 10 to 40° C., optionally from 20 to 30° C. It has been found that when a second fluid medium as described herein is passed down the intermediate channel (with the first fluid medium being passed down the other channels as described herein), a layered body is formed that has an intermediate layer comprising a gas.

The environment into which the fluid mediums pass into when exiting the channels may or may not contain a gas. Preferably, the environment into which the fluid mediums pass into when exiting the channels contains a gas, which may comprise a gas selected from nitrogen, oxygen, and a gas from Group 18 of the periodic table. The gas from Group 18 of the periodic table may be selected from helium, neon and argon. The environment into which the fluid mediums pass into when exiting the channels may contain air.

The environment into which the fluid mediums pass into when exiting the channels may contain a gas and be at a pressure of from 80 kPa to 120 kPa, optionally 90 to 110 kPa, optionally 95 to 105 kPa, optionally around standard pressure (101.325 kPa).

The volatile liquid may be selected from a non-polar liquid, a polar aprotic liquid, and polar protic solvents. Preferably, the volatile liquid comprises or is a perhalocarbon, most preferably a perfluorocarbon. Perhalocarbons are organic compounds consisting of carbon and halogen atoms. Perfluorocarbons are organic compounds consisting of carbon and fluorine atoms. Preferably the perhalocarbon, e.g. the perfluorocarbon, contains 10 carbon atoms or less, optionally 9 carbons atoms or less, optionally 8 carbons atoms or less, optionally 7 carbons or less, optionally 6 carbons or less, optionally 5 carbons or less, optionally 4 carbons or less. Preferably the perhalocarbon, e.g. the perfluorocarbon, contains 3 or more carbon atoms, optionally 4 or more carbon atoms. The perfluorocarbon may be selected from, but is not limited to, octafluoropropane, perfluorohexane, perfluoropentane, and perfluorodecalin.

The volatile liquid may comprise a halogenated hydrocarbon, which may be selected from, but is not limited to, a halogenated alkane, halogenated alkene and halogenated alkyne. The hydrocarbon may be branched or linear, and optionally substituted with one or more substituents other than a halogen. The halogenated hydrocarbon may have one or more halogens on each molecule, which may be selected from fluorine, chlorine, bromine and iodine. The halogenated hydrocarbon is preferably a fluoroalkyl. The halogenated hydrocarbon may contain 10 carbons or less, optionally 9 carbons atoms or less, optionally 8 carbons atoms or less, optionally 7 carbons or less, optionally 6 carbons or less, optionally 5 carbons or less, optionally 4 carbons or less. The halogenated hydrocarbon may contain 3 or more carbon atoms, optionally 4 or more carbon atoms.

Optionally, the volatile liquid comprises a heterofluoroalkyl. Examples of heterofluoroalkyls include, but are not limited to, methoxynonafluorobutane and ethoxynonofl uorobutane.

The volatile solvent may comprise an organic solvent selected from, but not limited to, ethanol, acetone, ethyl acetate, acetates, alcohol, ether, aliphatic, aromatic hydrocarbons, chlorinated hydrocarbons, ketones and chloroform.

The second fluid medium preferably has a dynamic viscosity of 1.3 mPa·s or less, optionally a dynamic viscosity of 1.2 mPa·s or less, optionally a dynamic viscosity of 1.1 mPa·s or less. The dynamic viscosity is measured at standard temperature (25° C.) and pressure (101.325 kPa). Dynamic viscosity values can be measured according to a standard method known to those skilled in the art, for example by using a U-tube viscometer or a rotational viscometer, such as a commercially available VISCOEASY rotational viscometer. Ethanol may used as a calibrating medium in the relevant measurement equipment, if necessary.

The second fluid medium preferably has a surface tension of 20 mNm⁻¹ or less, optionally 18 mNm⁻¹ or less, optionally 15 mNm⁻¹ or less, optionally 12 mNm⁻¹ or less. The surface tension of the second fluid medium is measured at standard temperature (25° C.) and pressure (101.325 kPa). Surface tension can be measured according to a standard method known to those skilled in the art, for example by using a tensiometer, e.g. a commercially available Kruss Tensiometer. Ethanol may used as a calibrating medium in the relevant measurement equipment, if necessary.

The conductivity of the second fluid medium is preferably 1×10⁻⁸ Sm⁻¹ or less, optionally 1×10⁻⁹ Sm⁻¹ or less, optionally 1×10⁻¹⁰ Sm⁻¹ or less, optionally 1×10⁻¹¹ Sm⁻¹ or less. The conductivity of the second fluid medium is measured at standard temperature (25° C.) and pressure (101.325 kPa). Conductivity can be measured according to a standard method known to those skilled in the art, for example by using a conductivity probe, such as the commercially available H1-8733 conductivity probe, available from Sigma-Aldrich. Ethanol may used as a calibrating medium in the relevant measurement equipment, if necessary.

Preferably, the first and second fluid mediums are immiscible. Optionally the volatile liquid has a solubility in the liquid of the first fluid medium of 100 ppm or less, optionally 50 ppm or less, optionally 20 ppm or less, measured at standard temperature (25° C.) and pressure (101.325 kPa). In an embodiment, the first fluid medium comprises a non-halogenated organic solvent and the second fluid medium comprises a perhalocarbon and/or a halogenated hydrocarbon. In an embodiment, the first fluid medium comprises a non-halogenated organic solvent and the second fluid medium comprises a perfluorocarbon and/or a halogenated hydrocarbon having one or more fluorines on each molecule. The non-halogenated organic solvent may be selected from, but is not limited to, an aprotic solvent and a protic solvent. The non-halogenated organic solvent may be selected from ethanol, acetone, ethyl acetate, acetates, alcohol, ether, aliphatic, aromatic hydrocarbons, chlorinated hydrocarbons, ketones and chloroform. In an embodiment, the first fluid medium comprises a non-halogenated organic solvent and a polymer, and optionally the second fluid medium comprises a perfluorocarbon and/or a halogenated hydrocarbon.

The method for producing the particles may be carried out, such that there is little or no lighting in the room in which the particles are produced.

### EXAMPLES

Example Particles Composed of PLGA Hydrolysable Polymer and Nitrofurantoin Active Agent for the Treatment of a Urinary Tract Infection or Intracellular Bacterial Infection. Apparatus for Synthesising Example Particles

Particles were synthesised by electrohydrodynamic processing, using a custom-made climate controlled Spraybase equipment, developed and designed at UCL by this team.

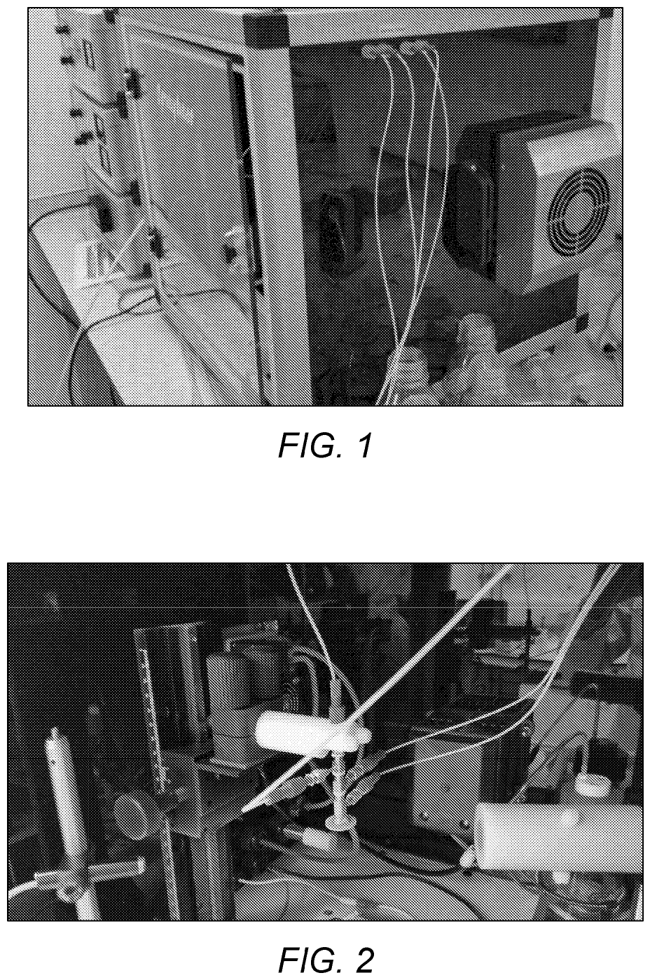

The fans (one is seen on the right of FIG. 1) are responsible for temperature control and do so via the Peltier effect. Glass bottles seen in the bottom of FIG. 1 contain the solutions to be sprayed. Tubing seen protruding from the top of the image connects the solutions in the glass bottles, to the quadra-axial needle inside the spraying chamber seen in FIG. 2. Control units for the applied pressure to each of the glass bottles as well as the applied voltage, temperature and humidity can be see towards the back and left of FIG. 1.

A laser pointer can be seen in the bottom left corner of FIG. 2 which is pointed at the tip of the quadra-axial needle seen in the centre of the image to illuminate the cone-jet. Towards the right, is a camera used for visualising the cone-jet.

Solutions to be processed were placed in a glass vial, each of which is placed in a glass bottle. A lid is attached to the bottle to create an airtight seal. The lid has an inlet for pressurised gas to be applied to the bottle, and an outlet whereby the tip of the tubing seen in FIG. 1 is inserted into the solution. When gas pressure is applied, the solutions are driven into the tubing and directed to the needle inside the spraying chamber. A potential difference on the scale of kilovolts is applied to the needle. As a result of balances in forces, the solutions emerging from the needle assume a cone shape with a jet emerging from the apex. The jet atomises the solutions which, upon solvent evaporation, produces polymer particles.

The solutions were pumped using a gas pressure driven system at a constant flow rate to the quadra axial needle system. After optimisation of the various controllable parameters which include potential difference, temperature, relative humidity, spraying distance, and applied pressure to drive liquid flow, the following parameters were used for processing of CapFuran (Example particle 1). A potential difference of 17 kV was applied to the needle. The solutions were sprayed between 16-20 degrees Celsius and 50-68% relative humidity for 10 minutes+/−1 minute. A spraying distance of between 150-200 mm was used. Pressures of between 0.015-0.033 bar were applied to each glass bottle in order to drive the liquids into tubing connected to the needles. After collection on a stainless steel plate, a plastic flat-ended spatula was used to scrape off the capsules. Capsules were transferred to glass vials for storage and transport at ambient temperature (17-22 degrees Celcius, measured using a data logger (Testo 174t)). The entire process from solution making to storage was carried out in a room with the lights switched off. Ambient lighting from external sources outside the room was sufficient carry out the process.

Example Particles and Formulation

For the preparation of solutions, all materials used were sourced as follows: Poly(lactic-co-glycolic acid)(PLGA)(copolymer 50:50, Resomer RG503H) was purchased from Evonik Industries AG (Essen, Germany). Pluronic F127, acetone, fluorescein isothiocyanate and nitrofurantoin were purchased from Sigma Aldrich (Poole, UK).

Example Particle 1:CapFuran

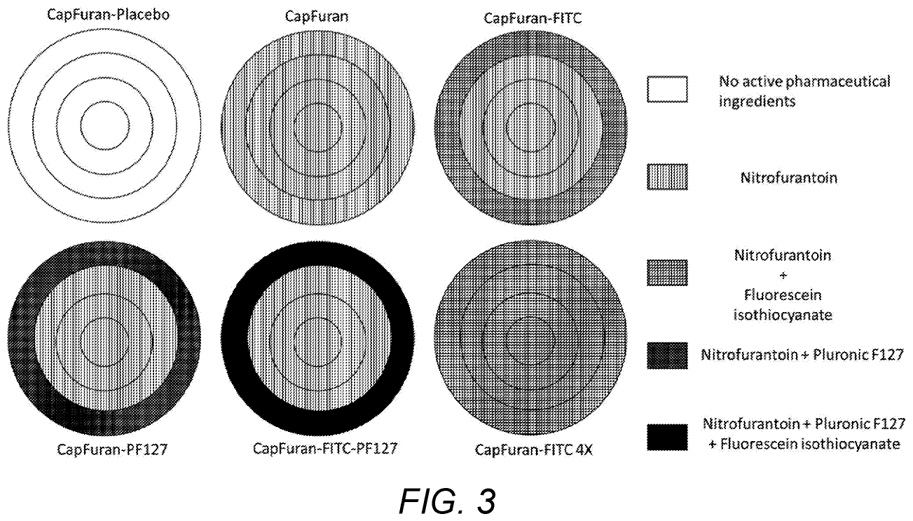

CapFuran contains the UTI first-line antibiotic nitrofurantoin (vertical shading) in all of its four layers as shown in FIG. 3. The solutions used to produce CapFuran are as follows:

PLGA was dissolved in acetone at 3 wt. %. Nitrofurantoin was dissolved in PLGA/acetone solution at 3.5 mg/ml. This solution is applied to each of the needle inlets.

Variants of CapFuran were also produced (Example Particles 2-5). As shown in FIG. 3 when a variant was produced, the solution applied to the outermost needle inlet contains the formulation relevant for the variant. In all cases, variants contain nitrofurantoin in all solutions used, with added components. These are underlined in the text. The solutions applied to the three inner needles remains the same as that detailed for CapFuran above, with the exception of Example Particle 5 and Reference Example Particle 6.

### Example Particle 2: CapFuran-FITC

CapFuran-FITC is a fluorescent tracker version containing PLGA, nitrofurantoin (vertical shading) and fluorescein isothiocyanate (horizontal shading). They are bright green under the correct wavelength of lights as expected, with similar size and resuspension profiles to CapFuran. These versions are used to test cell penetration, given that as mentioned above, urinary infections are often associated with bacterial reservoirs within the cytoplasm of bladder cells where normal antibiotics may not penetrate to a high enough concentration.

The solution used to produced CapFuran-FITC is as follows: Poly(lactic-co-glycolic acid)(PLGA)(copolymer 50:50, Resomer RG503H) was purchased from Evonik Industries AG (Essen, Germany). Acetone, fluorescein isothiocyanate and nitrofurantoin were purchased from Sigma Aldrich (Poole, UK). PLGA was dissolved in acetone at 30 mg/ml. Nitrofurantoin was dissolved in PLGA/acetone solution at 3.5 mg/ml. Fluoroscein isothiocyanate was dissolved in nitrofurantoin/PLGA/acetone solution at 1 mg/ml.

### Example Particle 3: CapFuran-PF127

CapFuran-PF127 contains an excipient, Pluronic F127 (also known as poloxomer 407) (dark shading) which acts as a biocompatible surfactant. This was added to improve the suspension characteristics of CapFuran. The solution used to produce CapFuran-PF127 is as follows:

Poly(lactic-co-glycolic acid)(PLGA)(copolymer 50:50, Resomer RG503H) was purchased from Evonik Industries AG (Essen, Germany). Acetone, fluorescein isothiocyanate and nitrofurantoin were purchased from Sigma Aldrich (Poole, UK). PLGA was dissolved in acetone at 30 mg/ml. Nitrofurantoin was dissolved in PLGA/acetone solution at 3.5 mg/ml. Pluronic F127 was dissolved in nitrofurantoin/PLGA/acetone solution at 3 mg/ml.

### Example Particle 4: CapFuran-FITC-PF127

CapFuran-FITC-PF127 contains both the fluorescent tracker and Pluronic F127 (black shading) along with the drug, and is used to test cell penetration of CapFuran-PF127. The solution used to produce CapFuran-FITC-PF127 is as follows: Poly(lactic-co-glycolic acid)(PLGA)(copolymer 50:50, Resomer RG503H) was purchased from Evonik Industries AG (Essen, Germany). Acetone, fluorescein isothiocyanate and nitrofurantoin were purchased from Sigma Aldrich (Poole, UK). PLGA was dissolved in acetone at 30 mg/ml. Nitrofurantoin was dissolved in PLGA/acetone solution at 3.5 mg/ml. Pluronic F127 was dissolved in nitrofurantoin/PLGA/acetone solution at 3 mg/ml. Fluoroscein isothiocyanate was dissolved in Pluronic F127/nitrofurantoin/PLGA/acetone solution at 1 mg/ml.

### Example Particle 5: CapFuran-FITC-4X

CapFuran-FITC-4X is an extra-bright fluorescent tracker version containing PLGA, nitrofurantoin (vertical shading) and fluorescein isothiocyanate (horizontal shading). Produced in a similar manner to Example Particle 2: CapFuran FITC, CapFuran-FITC-4X instead contains FITC in all layers of the particle.

### Reference Example Particle 6: CapFuran-Placebo

A version was also produced that contains only PLGA (“CapFuran-Placebo” (FIG. 3—no shading), which serves as an important negative control to assess whether the polymer base has any biological effect on its own. The solution used to produce CapFuran-Placebo is as follows:

PLGA was dissolved in acetone at 30 mg/ml. This solution is applied to each of the needle inlets.

Methods to Determine Particle Morphology and Size Distribution

Samples were scraped off the stainless-steel plates after processing and transferred onto a glass slide. A second slide was placed on top of the first and moved to spread the capsules evenly. Optical micrographs were taken with a camera (Micropublisher 3.3 RTV, 3.3 megapixel CCD Color-Bayer Mosaic, Real Time Viewing camera, MediaCybernetics, Marlow, UK) fixed to an optical microscope (Nikon Eclipse ME 600, Nikon, Japan). Samples on glass slides were gold coated using an ion sputter coater (Quorum Q150R ES) for 90 seconds at 20 mA before scanning electron micrographs were taken using a scanning electron microscope (JEOL JSM-6301F field emission scanning electron microscope, SEM). Particle sizes were measured using the imaging software, ImageJ (NIH).

Methods to Determine Feasibility of Terminal Sterilisation

Samples of CapFuran were sterilised (S-CapFuran) with 20 kGy of gamma radiation (Steris Ltd). After irradiation, CapFuran was analysed with SEM, FTIR, Raman Spectroscopy and shaking broth cultures.

FTIR analysis was performed via Attenuated Total Reflection Fourier Transform Infrared spectroscopy (ATR-FTIR) measurements (Bruker Vertex 90 spectrometer), and spectrographs were interpreted using OPUS Viewer version 6.5 software. The resolution was 4 cm⁻¹ and the scan count was 16, over 4000-500 cm⁻¹ at ambient temperature.

Raman Spectroscopy was performed using a Renishaw-2000 laser Raman spectroscopy system at a wavelength of 785 nm and total exposure time of 10 seconds.

Methods to Determine Antibacterial Activity in Shaking Broth Cultures

The protocol for the antibacterial assay was similar to that previously reported (Labbaf et al., 2013). Briefly, liquid cultures of bacteria were grown and co-cultured with various capsule or control conditions in a shaking incubator and samples withdrawn at various time points to enumerate bacterial growth post-treatment on agar plates. If unspecified, the bacterial strain used was E. faecalis, a strain derived from a patient with chronic UTI (Horsley et al., 2013), which is a common uropathogen in such patients, as well as in hospital-acquired infections. The maximum concentration of capsules used was the equivalent of 200 ug/ml of nitrofurantoin according to the manufacturer's pharmacological information, which is the average expected concentration reached in the bladder following standard oral delivery.

One single colony of E. faecalis growing on CPS Elite chromogenic agar plates (bioMérieux) for 24 hours in an aerobic incubator at 37 degrees was resuspended in 5 ml of tryptic soy broth (Sigma) overnight in the 37° C. shaking incubator. Comparing with a 0.5 MacFarlane standard, bacterial broth was resuspended in CnT Prime bladder epithelia media (CellNTec). It was then further diluted 1:250 in the same media (concentration of approximately 4×10⁷ colony-forming units [CFU]/mL) and this was added to the capsules and drug solutions in 15 ml Falcon tubes. Various dilutions of treatments were used (see Figures for details on a per experiment basis). Depending on the experiment, controls included: free nitrofurantoin and CapFuran-Placebo (“blank” capsules containing only polymer). When bacterial suspension is added to the treatment in a 1 to 1 ratio, concentrations are therefore halved. For example, to achieve 200 ug/mL capsules, the Falcon tube would initially contain 400 ug/mL. Shaking orbital incubation was conducted overnight (typically 16-24 hours) in darkened conditions at 37 degrees with loose caps to allow aerobic respiration. Enumeration was conducted by withdrawing 25 uL of sample and plating this on a quadrant of CPS Elite or Columbia blood agar (Oxoid) and allowing to grow overnight; colonies were scored by counting and back-calculating the total amount of viable “colony-forming units” (CFU) in the original Falcon tube. A quadrant containing too many colonies to count was arbitrarily estimated at 600 CFU, and a quadrant growing a seamless lawn of bacteria was set at 1000.

Cell Culture Methods

Commercially available human bladder epithelial cells (HBLAK, CellNTec), which are a spontaneously immortalised, non-transformed derivative of progenitor cells that retain the ability to differentiate, were supplied in frozen aliquots containing ˜5×10⁵ cells at passage 2 and ˜0.5×10⁵ at passage 25 respectively. The progenitor cells had been isolated from bladder trigone biopsies from male patients undergoing surgery for benign prostatic hyperplasia. HBLAK cells were used up until passage 40-50.

Thawed cells were seeded (˜300 cell clumps/cm²) into pre-warmed and equilibrated low-calcium, high-bovine pituitary extract, primary epithelial medium (CnT-Prime, Cell N Tec) in 9 cm polystyrene dishes and incubated at 37° C. in a humidified incubator under 5% CO₂. Culture medium was replaced after overnight incubation to remove residual dimethyl sulfoxide (DMSO). Antibiotics were not added to culture medium at any point due to adverse effects on cytodifferentiation, metabolism and morphology. Furthermore, trypsin is known to damage primary cells; therefore, Accutase solution (Innovative Cell Technologies) was used to detach cells at all stages of experimentation. Cells were allowed to expand to ˜70% confluency before freezing batches of cells at a density of ˜1×10⁶ cells/ml in defined freezing medium (CnT-CRYO-50, Cell N Tec) in preparation for later experiments. Cells were not allowed to become fully confluent during cell expansion in an effort to maintain a proliferative phenotype.

The three-dimensional bladder organoid model has been described (Horsley et al, 2017, https://doi.org/10.1101/152033). Briefly, organoids were created as follows. In preparation for organotypic culture, previously frozen cells were thawed and expanded on 9 cm culture dishes as above. Once 70-80% confluent, the cells were washed briefly with calcium- and magnesium-free phosphate buffered saline (PBS, Sigma-Aldrich) and incubated at 37° C. in ˜3 ml of pre-warmed Accutase solution for 2-5 min. The dishes were lightly tapped and detached cells re-suspended in 7 ml of warm CnT-Prime. After centrifugation at 200×g for 5 min, the supernatant was removed and the pellet re-suspended in fresh CnT-Prime. This cell suspension was counted whilst allowing the cells to equilibrate for 3 min at room temperature. 2×10⁵ cells in 400 μl of CnT-Prime (internal medium) were added to 6 12 mm 0.4 μm pore polycarbonate filter (PCF) inserts (Millipore) standing in 6 cm culture dishes containing ˜3 ml of fresh pre-warmed CnT-Prime medium (external medium, level with insert filters). A further 8 ml of CnT-Prime medium was added to the 6 cm dish (external to the filter inserts) until internal and external fluid levels were the same.

The 3D culture inserts were incubated for 3-5 days until 100% confluent. Confluency was determined through the fluorescent staining of 1 insert and visualisation under epi-fluorescence microscopy (see section below). Once deemed confluent, internal and external medium was removed and replaced with low-BPE, calcium-rich (1.2 mM) differentiation barrier medium (CnT-Prime-3D, Cell N Tec) to promote differentiation. Subsequent to overnight incubation, the internal medium (apical surface of cell culture) was removed and replaced with filter-sterilised human urine pooled from healthy volunteers of both genders to aid terminal differentiation into umbrella cells. The external CnT-Prime-3D medium and the internal human urine were replaced every 3 days and the culture incubated for 14-24 days at 37° C. in 5% CO₂.

Methods to Determine Cellular Uptake of Cargo

For two-dimensional monolayer assays, HBLAK cells were seeded onto 8-well glass chamber slides (LabTek) that had been pre-coated for 1 hr at 37 degrees C. with fibronectin solution (Sigma, in PBS at 0.1 mg/mL), grown until about 100% confluent and treated with capsules loaded with the fluorescent green dye FITC (CapFuran-FITC), or with blank capsules (CapFuran-Placebo) as a control, for the indicated doses and times (see Figures for details). After the treatment time, cells were washed briefly with PBS, then fixed with freshly diluted 4% formaldehyde in PBS for 20 min. When permeabilization was required, this was done in 0.2% Triton-X100 (Sigma-Aldrich) in PBS for 15 minutes at RT followed by a single wash with PBS. The following staining was performed in PBS for 1 hour at RT depending on the experiment: TRITC or AlexaFluor-633-conjugated phalloidin (0.6 μg/ml)(Sigma-Aldrich), to label filamentous actin; and the DNA stain 4″,6-diamidino-2-phenylindole, (DAPI, 1 μg/μl; Sigma-Aldrich). The labelling solution was gently aspirated and the cells washed 5 times in PBS before mounting. Lab-Tek slide wells and gaskets were carefully removed prior to the addition of FluorSave (Calbiochem) and a coverslip coverslip fixed in place with clear nail varnish.

When the uptake experiment was performed on organoids to look at uptake in a three-dimensional tissue, the incubation took place in situ in the transwells, and prior to staining, filter inserts were carefully transferred to 8-well plates (Nunc) and submerged in 4% methanol-free formaldehyde (Thermo Scientific, Fisher Scientific) in PBS overnight at 4° C. After fixation, the filter inserts were kept at 4° C. in 1% formaldehyde in sealed containers in preparation for processing. Filters on which the tissue was affixed were removed with forceps, places in FluorSave, and a coverslip fixed in place with clear nail varnish.

We performed epi-fluorescence microscopy on an Olympus CX-41 upright microscope, and confocal laser scanning microscopy on Leica SP5 and SP2 microscopes. For timelapse videomicroscopy, Images were taken using a fully-motorised Leica SP8 laser scanning confocal microscope equipped with hybrid detectors and hardware-based autofocus. Leica Application Suite X (LASX, version 3.5.2.18963) with Lightning super-resolution module was used to control the microscope and analyze data. Cells were grown to confluency on 35 mm live-imaging dishes (μ-Dish, Ibidi) inside a stage-top incubator receiving 0.35 l/min of pre-mixed gas containing 5% CO₂ within a fully-enclosed microscope cabinet heated to 37 C. Live Z-stacks comprising of 10 slices (Z-step of 1.93 μm) at 16 bit at a resolution of 2880×2880 were taken every 90 seconds for a total duration of 30 minutes. For both still and live imaging, images were processed and analysed using Infinity Capture and Analyze V6.2.0, ImageJ 1.50 h 50 and the Leica Application Suite, Advanced Fluorescence 3.1.0 build 8587 Software.

Cell Toxicity Assay

A colorimetric lactose dehydrogenase (LDH) assay kit (Thermo Scientific) was used to measure potential cell damage from CapFuran or nitrofurantoin. The procedure was carried out as directed by the manufacturer. HBLAK-derived bladder organoids were grown for 14 days then exposed to 1000 μl of culture media (control) or 1000 μl of culture media containing CapFuran 1 mg/ml; CapFuran 2 mg/ml; 100 μg/ml unencapsulated nitrofurantoin; 200 μg/ml Nitrofurantoin; 100 μl of 10× lysis buffer (maximum LDH control); or culture medium containing 10% ultra-pure water (to measure spontaneous LDH release). Experiments were carried out in triplicates.

All organoids were subsequently incubated for 60 minutes at 37° C. in 5% CO₂. After incubation, 50 μl of medium from the apical chamber of each treated organoid was transferred to 3 wells of a flat-bottomed 6-well plate (Corning). 50 μl of reaction buffer (lactate, NAD⁺, tetrazolium salt (INT)) was then added to each well and gently mixed before incubating the plate at room temperature for 30 minutes in darkness. The reaction was then halted by adding 50 μl of stop solution (0.16M sulfuric acid) to each well.

To quantify the amount of LDH released, the 96 well plate was read using a colorimetric sphectrophotometer (Biochrom EZ Read 400) at an absorbance of 492 nm and 650 nm. Microsoft Excel was used to subtract the background reading from the LDH reading before calculating cytotoxicity in % using the following formula:

$$\% Cytotoxicity = \frac{\begin{matrix}Treatment associated LDH release - \\ Spontaneous LDH release\end{matrix}}{\begin{matrix}Maximum LDH activity - \\ Spontaneous LDH release\end{matrix}} \times 100$$

Antibiotic Protection Assay Methods

The purpose of the antibiotic protection assay is to assess the ability of a given treatment to kill intracellular bacteria. This classic, commonly used and validated test (see Mulvey et al., Infect Immun. 2001: doi 10.1128/IA1.69.7.4572-4579.2001) used to understand the dynamics of intracellular pathogens, involves killing all extracellular bacteria using an antibiotic, such as gentamicin and/or vancomycin, that cannot pass through the cell membrane, before washing and lysing the cells with detergent and enumerating any viable bacteria that have been “protected” inside. In the case of these experiments, we treat with CapFuran, culture media or nitrofurantoin after the antibiotic treatment for a period before the lysis step to evaluate intracellular killing activity.

A colony of E. faecalis was added to 5 ml of Tryptic Soy Broth (and grown overnight in an orbital shaker at 37 degrees). 100 μl of bacteria were resuspended in 5 ml of fresh Tryptic Soy Broth for 3 hours and an optical density corresponding to 0.4 (corresponding to 2×10⁹ CFU) was used for the assay.

HBLAK cells (2×10⁵ cells) were seeded onto 12 mm 0.4 μm polycarbonate filter inserts and organoids were grown as described above. For infection, 500 μl of CNT 3D prime media and bacteria were added to the inserts at a multiplicity of infection (MOI) of 10 and the cells were incubated at 37° C. in 5% CO₂ and left overnight.

After washing with PBS solution once, all 5 inserts were treated with 2 hours of gentamicin (150 μg/ml) and (vancomycin 10 μg/ml) in CNT 3D prime media solution. The supernatants of two of the inserts were then plated on Tryptic Soy Agar (TSA) plates in duplicates. These two inserts were then washed with PBS once followed by the addition of 500 μl 1% Triton-X100 in PBS for 10 minutes at room temperature to lyse and liberate viable intracellular bacteria. Supernatant solutions were plated as described above. These served as controls to show that bacterial invasion occurred.

The remaining 3 inserts, having been treated with 2 hours of gentamicin (150 μg/ml) and vancomycin (10 μg/ml), were then washed with PBS once followed by the addition of 10 μg/ml gentamicin (bacteriostatic dosage) and left overnight at 37° C. in 5% CO₂ to allow any intracellular bacteria to flourish.

The following day, the inserts were washed once with PBS (supernatants were plated as above) and assigned treatment with 2 mg/ml CapFuran capsules, 200 μg/ml nitrofurantoin CNT 3D prime media solution (“mock”) for 2 hours. The supernatants for each of the conditions were plated.

The cells were then lysed with 1% Triton-X100 and plated as described above. For plating of bacteria, inoculum or supernatants were diluted to 1×10⁹ in PBS solution using a 96 well plate. 10 ul of each dilution was cultured on TSA plates in an incubator at 37° C. for 24 hours. To establish the correct CFU (colony forming units) per mL, the mean bacterial count from the duplicate samples were multiplied out of its serial dilution.

In Vitro Biofilm Growth Methods

Two substratum conditions for biofilm growth were used in these experiments: (i) porous polycarbonate membranes (PPM); and (ii) Lab-Tek glass chamber slides coated in a layer of fixed human uroepithelial cells (HCLT). Porous polycarbonate membranes with a 12 mm diameter and 0.4 μm pore size (same as organoid substratum described above) were suspended from Transwell® permeable supports in a 12 well plate (Corning Costar 3401, NY, USA). HBLAK human uroepithelial cells (CELLnTEC, UK) were maintained at 37° C., 5% CO₂, in CnT Prime medium (CELLnTEC, UK) that was changed every two days. When cells reached 70-80% confluency, they were resuspended onto an eight well glass Lab-Tek™ (Thermo Fisher Scientific 154534, UK) pretreated with 0.5% fibronectin from bovine plasma (Merk F1141-5MG, UK). The correct concentration of fibronectin was achieved using Phosphate-buffered saline (PBS) (without calcium and magnesium) pH 7.2 (1×) (20012-027 from Gibco®, UK), of which 200 μl was added to each well and then incubated at 37° C., 5% CO₂ for one hour. Cells were resuspended according to CELLnTEC cultivation protocol, which involved washing the cells once with PBS, followed by cell detachment using 1 ml of Accutase (CELLnTEC, UK). Cells were incubated for approximately five minutes at 37° C., 5% CO₂ until they showed signs of detachment under the light microscope, whereupon Accutase was deactivated by the addition of 2.5 ml of CnT Prime medium which had been stored at 37° C., 5% CO₂ 30 minutes prior to use. The cell solution was transferred to a 15 ml Falcon tube and centrifuged at 1000 rpm for five minutes. Next, the supernatant was aspirated and replaced with 5 ml of fresh CnT Prime. 150 uL of the resultant cell solution was then added to each well of the pretreated LabTek containing 250 μl of CnT Prime. Subsequently, the LabTeks were incubated at 37° C., 5% CO₂ until at least 50% confluency was achieved. Cells were fixed using a solution of 4% methanol-free formaldehyde (Sigma-Aldrich F8775, UK) in PBS solution for 20 minutes, at room temperature, in the dark. The formaldehyde was then aspirated and the cells were washed three times using 400 μl PBS. Finally, 400 μl of PBS was added to each well and the LabTeks were wrapped in parafilm and stored at 4° C. until use.

For each substratum, uropathogenic E. faecalis, which had been previously obtained from patients and stored in glycerol at −80° C. on Microbank™ microporous ceramic beads, was maintained on CPS Elite (CPSE) plates (418284 from bioMerieux, UK) at 4° C. A single E. faecalis colony was then resuspended in 5 ml of Tryptic Soy Broth (TSB)(Merck 22092, UK) and incubated shaking at 37° C. for a minimum of four hours to achieve a stationary phase of 1×10⁹ bacterial cells per ml. At this point, TSB supplemented with 1% (D-(+)-Glucose)(Merck G8270, UK) was prepared for each substratum and bacterial culture added as follows. For the PPM substratum, each well of the PPM 12 well plate contains an inner and outer well which requires 1500 μl of medium and 500 μl of bacterial culture solution respectively. The contents of the inner and outer wells must be maintained at these levels to avoid the movement of substances between the two. Therefore, 500 μl of E. faecalis TSB solution, which had been further diluted with TSB to achieve a concentration of 2.5×10⁶ bacterial cells per ml, was added to each inside well. Next, 1500 μl of 1% glucose TSB solutions were prepared and added to the outer wells. For the HCLT model, TSB of 1% glucose concentrations were prepared. 399 microlitres of each solution was added to a well of the Lab-Tek™, followed by the addition of 1 μl of E. faecalis TSB solution to achieve a concentration of approximately 2.5×10⁶ bacterial cells per ml in each well. Finally, both the PPM 12 well plate and HCLT were incubated at 37° C., 5% CO₂ for 24 hours

Fluorescent Anti-Biofilm Assay Methods

Biofilms were prepared as described above, using both substratum in addition to 1% glucose TSB solution and 24 hour incubation. Biofilms were then washed once with PBS to remove planktonic bacteria and treated with one of four different conditions: TSB alone (mock treatment); 0.2 mg/ml Nitrofurantoin (free drug); 2.5 mg/ml CapFuran-Placebo; or 2.5 mg/mL CapFuran-FITC.

For HCLT biofilms, each well received 400 μl of each treatment. Dilutions of each treatment were prepared using TSB. For PPM biofilms, each well received 500 μl of treatment. Biofilms were then incubated shaking at 37° C., 5% CO₂ for two hours in the dark. After removal from the incubator, each treatment was aspirated and biofilms were washed three times with PBS to remove residual treatments. Biofilms were then fluorescently stained and mounted.

Upon removal from the incubator, the contents of each HCLT well and inner well of the PPM 12 well plate were removed and washed once with 200 μl and 500 μl of PBS respectively to remove any planktonic bacteria. First, biofilms were stained with 5-Cyano-2,3-di-(p-tolyl)tetrazolium chloride (CTC) (Merck 94498, UK). A stock solution of 0.05M CTC was prepared using distilled water and stored at 4° C. in the dark. Upon biofilm staining, a 0.005M CTC solution was produced using 1% glucose PBS solution. 100 μl and 500 μl of 0.005M CTC solution was then added to each well of the HCLT and inner well of the PPM 12 well plate respectively, and left at room temperature in the dark for one hour. The CTC solution was then aspirated and each biofilm was washed, as above, three times. Each biofilm was fixed in 4% formaldehyde PBS solution for 20 minutes, at room temperature, in the dark. The formaldehyde solution was then aspirated and biofilms were washed three times with PBS. Biofilms were then stained with DAPI and WGA and mounted as described above for bladder cells.

Confocal laser scanning microscopy (CLSM) of treated and fluorescently stained HCLT biofilms was performed on a Leica SP2 microscope. Samples were excited with laser lines 358 nm, 450 nm and 494 nm. CLSM images of PPM were acquired using a Zeiss LSM 510 Meta microscope using the 405, 488 and 543 nm laser lines. Z-series data were collected using a 63× Plan Apo oil immersion objective and confocal sections collected at 0.29 [micrometer] intervals. Each is 40-60 confocal sections. Quantitative and qualitative image analysis of biofilm architecture from confocal Z-stack images was performed using image J with Comstat 2.1. The biomass of each channel was calculated by first manually adjusting the threshold intensity to reduce background noise. The images were then analysed using the histogram function which provided a list of the number of pixels above the set threshold (Pₜₕᵣₑₛₕ). Subsequently, this was used, along with the x, y and z dimensions of each voxel to calculate the biomass (μm³/μm²) as follows:

Biomass=Pₜₕᵣₑₛₕ*X*y*z/x*y

The DAPI:CTC biomass ratio was calculated by dividing the DAPI biomass by the CTC biomass.

3D images for the assessment of capsule penetration and generation of Z position data were done using Imarls 8.4.1×64. For both the FITC (green) and DAPI (blue) channels, a smooth surface representing the absolute intensity for each signal was produced using a surface detail of 0.465 μm. Threshold and quality values were set objectively and split touching objects enabled. The statistical tool was used to produce the Z positions of each defined object.

Z position data analysis was performed in Excel (Microsoft, 2010) using the histogram function.

Biofilm Killing Assays

A colony of E. faecalis was added to 5 ml of Tryptic Soy Broth (and grown overnight in an orbital shaker at 37° C.). A 1:250 solution of bacteria supplemented with fresh TSB and 1% glucose was made and aliquoted into 96 well plates. Biofilms were allowed to form by incubating the plates in a humidified chamber at 37° C. for 48 hours in an orbital shaker (150 RPM). After 48 hours, the supernatant solution was aspirated before either treating the wells with 2 mg/ml CapFuran; 2 mg/ml CapFuran-Placebo; fresh TSB only; or 200 μg/ml unencapsulated nitrofurantoin, all resuspended in TSB solution. After overnight treatment, the supernatant solutions were plated in duplicates in Tryptic Soy Agar plates to enumerate the numbers of planktonic bacteria not incorporated in the biofilm.

The 96 well plates were then washed gently with PBS four times before the biofilms were mechanically disrupted using a pipette tip. Bacterial enumeration of the biofilm was carried out in duplicates by spot-plating and enumeration on Tryptic Soy Agar plates.

Results

CapFuran and its Test Derivatives can be Readily Produced in a Reproducible Manner in a Format Amenable to Resuspension in Bio-Compatible Liquids

CapFuran (Example Particle 1), produced via electrohydrodynamic atomisation (also known as electrospraying), contains a combination of nitrofurantoin and poly(lactic-co-glycolic acid) (PLGA), is a biodegradable, porous microparticle, and can be readily resuspended in aqueous solution (e.g. phosphate-buffered saline, cell culture media) for dosing, which can be used for treatment of UTI. 10 mg of dry capsules contain approximately 0.9-1.1 mg of nitrofurantoin; so a concentration of 10 mg/ml of capsules should contain approximately 0.9-1.1 mg/ml of nitrofurantoin. The normal bladder dose in a patient taking oral nitrofurantoin typically reaches 200 micrograms/mL—so to achieve an approximately biosimilar amount, capsule experiments were run at 2.0 mg/mL dry weight solutions. Other concentrations were occasionally used; refer to figure legends and/or text for doses in all cases.

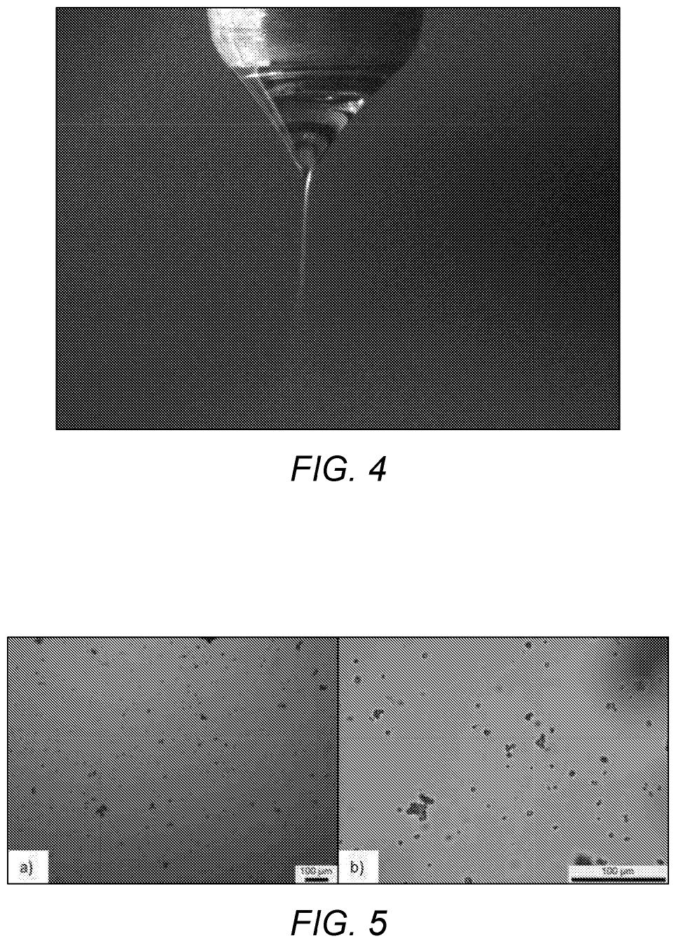

Spraying was carried out in the cone-jet regime as shown in FIG. 4. This is the most stable regime of electrospraying and produces the particles with the smallest size distribution.

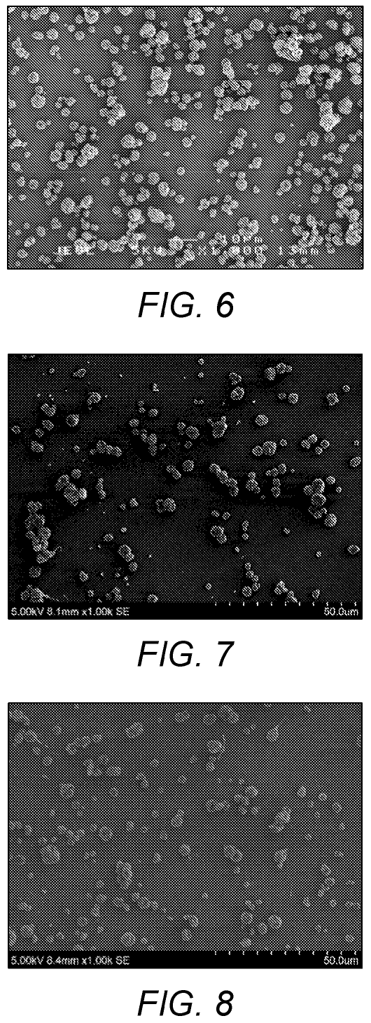

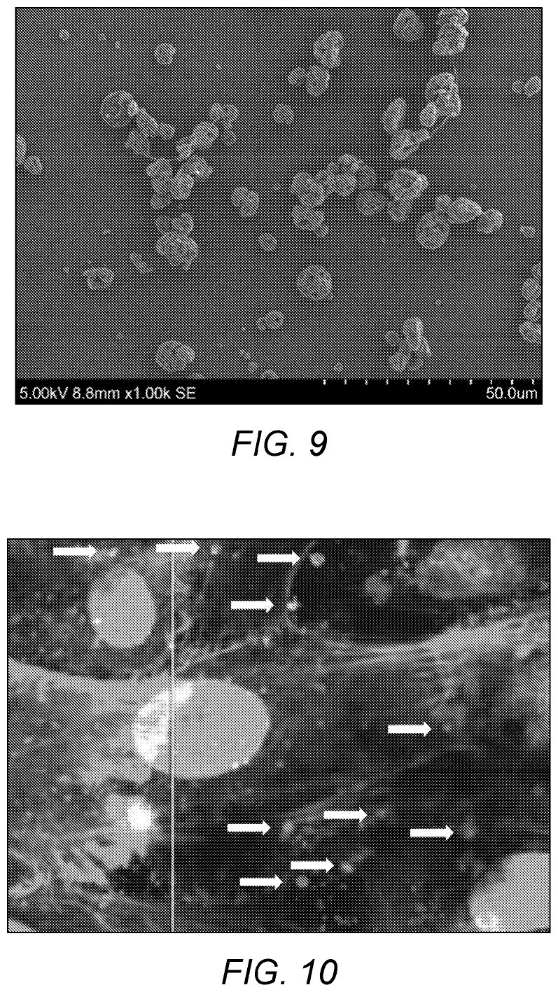

CapFuran was imaged by an optical microscope (Micropublisher 3.3 RTV, 3.3 megapixel CCD Colour-Bayer Mosaic, Real Time Viewing camera, Media Cybernetics, Marlow, UK) at 5× and 20× magnification (FIG. 5) and a SEM (JEOL JSM-6301F) (FIG. 6). CapFuran has the appearance of a yellow powder and as seen from FIG. 6, has a porous, approximately spherical morphology with a size distribution of 1-6 microns, (as assessed by quantifying SEM images), with a distribution as shown in Table 1 SEM images of other CapFuran formulations (e.g. CapFuran-FITC, CapFuran-PF127, CapFuran-FITC-PF127 are shown in FIGS. 7-9).

TABLE 1

| Diameter range in microns | Percent of population being in that range |
|---|---|
| 0-1 | 0 |
| 1-2 | 16 |
| 2-3 | 36 |
| 3-4 | 33 |
| 4-5 | 9 |
| 5-6 | 6 |

Fluorescent tracker versions containing PLGA and fluorescein isothiocyanate with and without the nitrofurantoin) are bright green under the correct wavelength of lights as expected, with similar size and resuspension profiles to the drug version. FIG. 10 shows a field of bladder cells onto which CapFuran-FITC has been added during treatment. The white arrows point out glowing green capsules. These versions are used to test cell penetration, as urinary infections are often associated with bacterial reservoirs within the cytoplasm of bladder cells where normal antibiotics cannot penetrate.

CapFuran can be Sterilised with 20 kGy of Gamma Radiation (S-CapFuran) with No Detectable Changes to the Chemical Bonds Present and No Significant Impact on its Efficacy Against Bacteria.

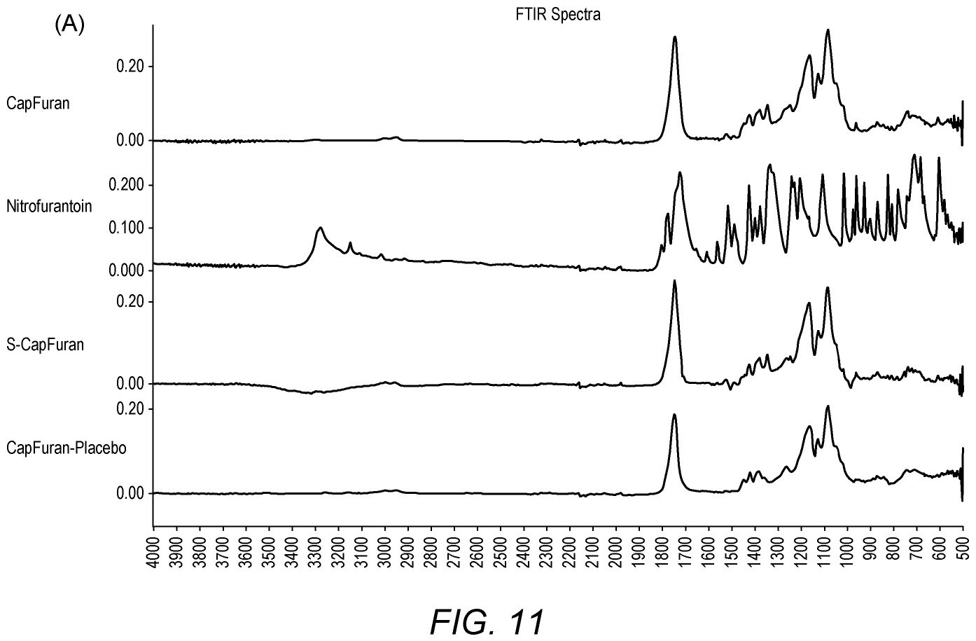

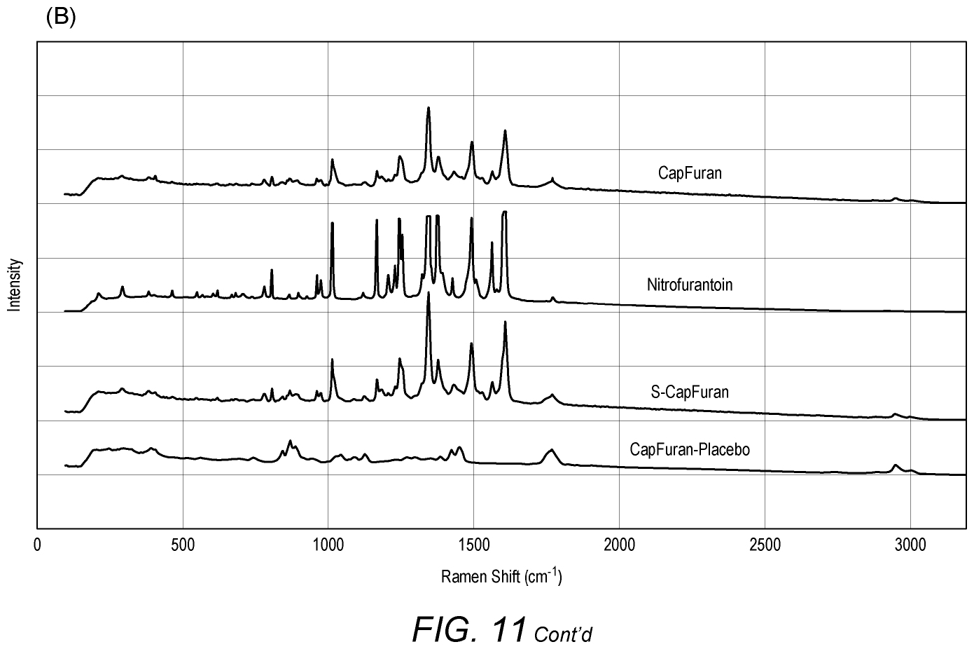

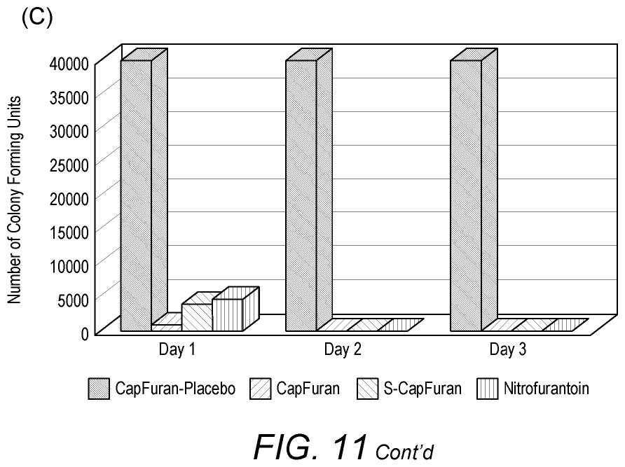

FIGS. 11A and 11B show FTIR and Raman spectra respectively comparing CapFuran, free nitrofurantoin, S-CapFuran and CapFuran-Placebo. FIG. 11A shows the FTIR spectra of CapFuran and S-CapFuran to be similar each other and to CapFuran-Placebo (which contains only PLGA). The major peaks are seen at approximately 1750 cm⁻¹ corresponding to the C═O carbonyl group, 1350-1420 cm⁻¹ corresponding to the C—H methyl groups and 1080, 1120 and 1170 corresponding to the C—O—C ester bonds. Only a few of the intense peaks seen in the spectra of nitrofurantoin were apparent in CapFuran or S-CapFuran spectra, suggesting the presence of PLGA masks the spectra of nitrofurantoin.

FIG. 11B shows the Raman spectra of CapFuran and S-CapFuran to be nearly identical to each other and displaying peaks seen in both nitrofurantoin and CapFuran-Placebo (PLGA). The majority of the peaks seen with nitrofurantoin are also seen in CapFuran and S-CapFuran with some key differences. The C—H peaks seen at around 2950 cm⁻¹ are absent in nitrofurantoin but present in CapFuran and S-CapFuran spectra. Three distinct peaks seen in the 800-900 cm⁻¹ range for nitrofurantoin are not seen or may be masked in the CapFuran and S-CapFuran spectra. The relative intensity of the twin peaks seen at 1350 and 1380 cm⁻¹ is altered in the CapFuran and S-CapFuran spectra, with the later peak at 1380 cm⁻¹ much less intense relative to the former 1350 cm⁻¹ peak.

FIG. 11C shows a killing assay with E. faecalis with CapFuran (Example Particle 1) and S-CapFuran. By day 2, both CapFuran and S-CapFuran have considerably reduced bacterial populations and by day 3, no living bacteria were present. This experiment shows that sterilisation has no effect on CapFuran's efficacy.

CapFuran-FITC can Reproducibly Enter Cultured Human Bladder Cells to Deliver Cargo in a Dose-Responsive Manner, with No Significant Toxicity and with 100% of the Cells Taking Up Dye at a Concentration of Polymer at an Oral-Similar Dose, at a Much Higher Efficiency than can Free FITC Itself

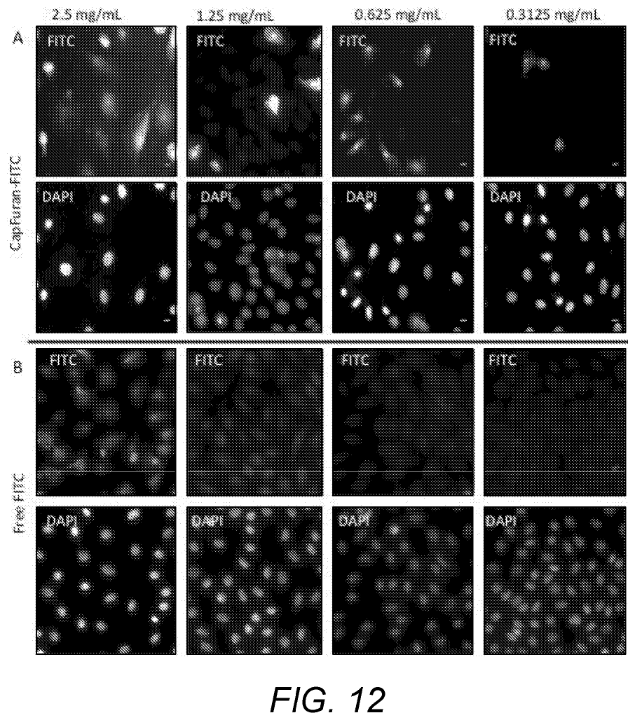

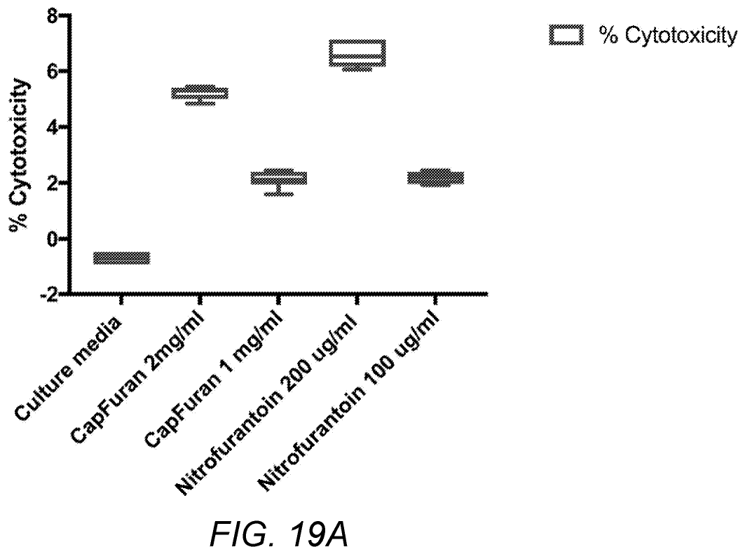

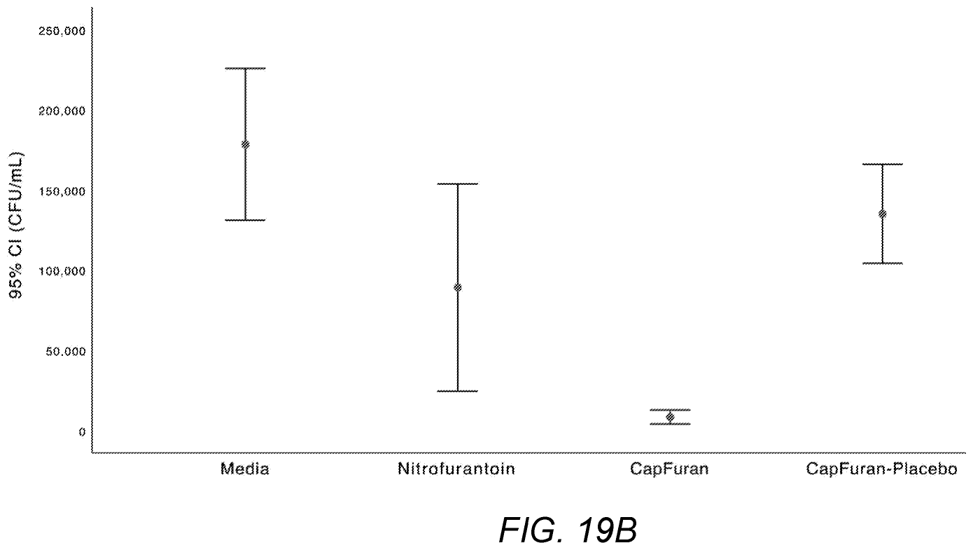

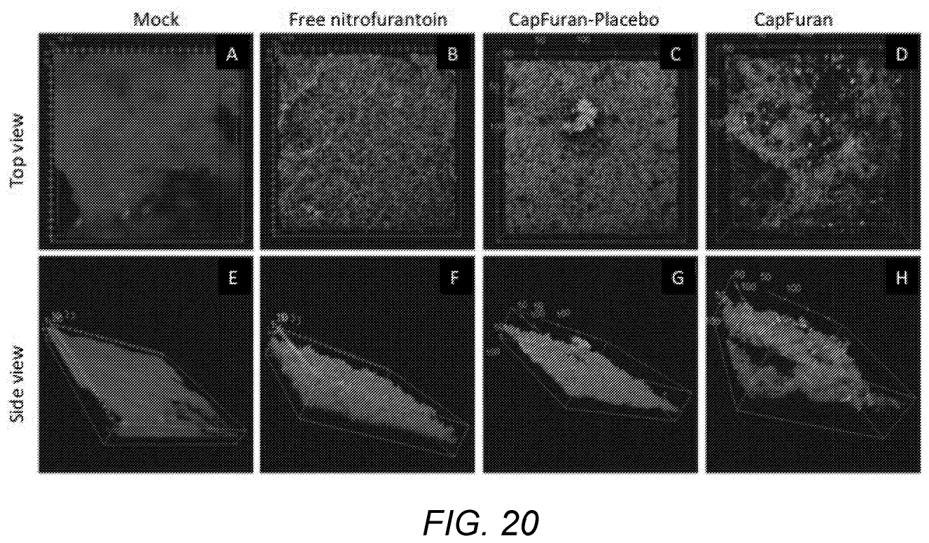

The FITC-loaded version of CapFuran (CapFuran-FITC, Example Particle 2) was used to assess cellular uptake and cargo delivery in cultured human bladder cells (HBLAK). As shown in FIG. 12A, 100% of cells take up the cargo after 2 hours of treatment at doses of 2.5 mg/mL. As the dose is systematically reduced below this, the number of positive cells decreases, showing that delivery is cell-specific and not merely the result of any free FITC that might have been released prior to docking and delivery. If uptake were due to free FITC diffusion into cells, it would be expected that all cells would be green but that its intensity would gradually decrease with dose. Indeed, as shown in FIG. 12B, free FITC at 2.5 mg/mL bestows a faint green staining to all cells but does not confer the bright green staining produced by delivery via CapFuran-FITC. Also, the gradual diminishment of signal in all cells as concentration is decreased is exactly what would be expected with free diffusion. In contrast, FIG. 12A clearly shows that at lower doses, only some cells take up the dye, but still glow brightly, which is expected from collision and uptake of a rare particle in some but not all cells. No apparent toxicity was observed after capsule treatment; all cells appear morphologically healthy. (This observation was confirmed quantitatively in the human organoid; see FIG. 19A, below). Some intact capsules can be seen outside of cells.

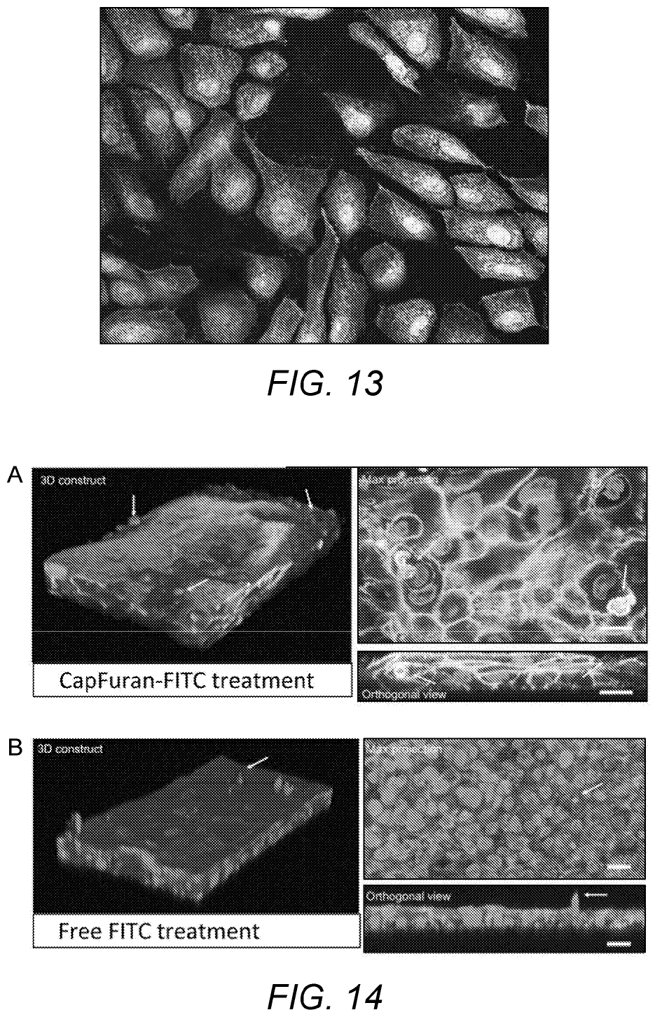

FIG. 13 demonstrates a close-up of cells that have taken up the green cargo after 2 hr; note that the dye is present in both the cytoplasm and nucleus as expected for its size. Echoing what is seen in FIG. 12A, the bright green appearance of positive cells suggested that the intracellular concentration of capsule-delivered FITC was much higher than that which would occur with free diffusion.

Taken together, these experiments show that CapFuran-FITC (Example Particle 2) can deliver cargo intracellularly to all cells in a culture, and can achieve an intracellular concentration much greater than that conferred by free diffusion, in a manner far surpassing our 2013 PMSQ prototype.

CapFuran-FITC can Penetrate a Three-Dimensional Human-Cell Derived Bladder Urothelial Organoid Model Through Multiple Layers of Cells Whereas Free FITC Cannot

The uptake experiment was repeated in a bespoke human urothelial organoid model, which should present a more formidable barrier to entry, since it elaborates asymmetric unit membrane plaques and an apical extracellular glycosaminoglycan layer (Horsley et al, 20182017).

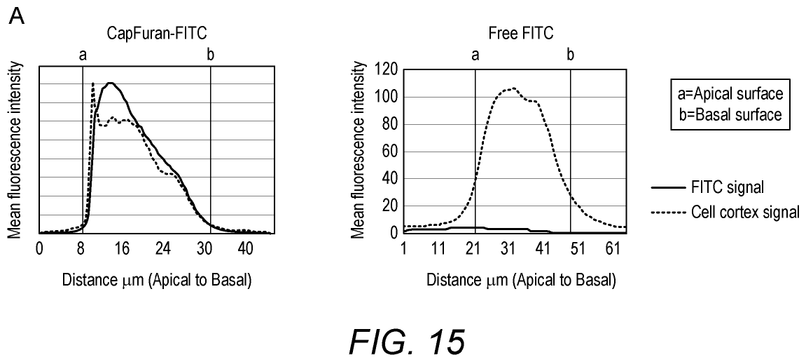

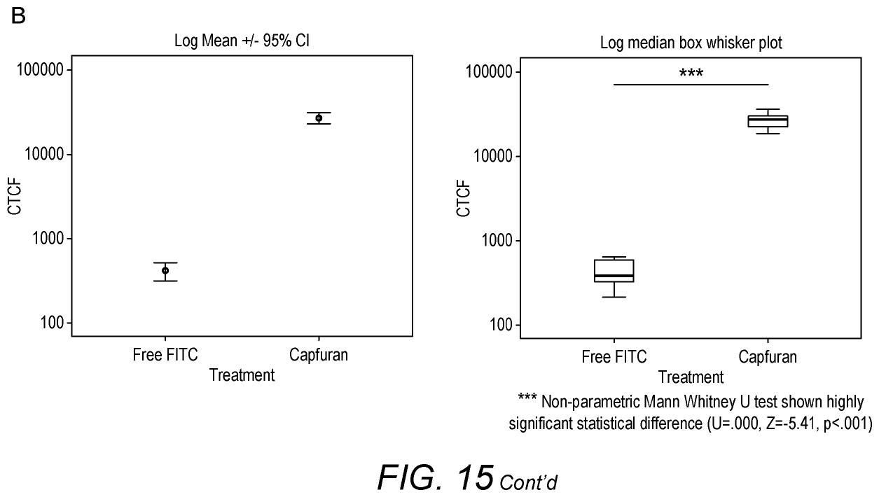

As shown in FIG. 14A, after two hours of treatment with CapFuran-FITC, the dye (grey haze) is distributed in all layers, showing penetration through multiple layers of cells. Penetration is most concentrated at the top cell later and becomes less concentrated towards the bottom. This result was confirmed by a pixel analysis of the green channel (FIG. 15A) showing mean fluorescence intensity from top (left) to bottom (right).

In the same experiment, similar organoids were challenged with free FITC at the same dose as what is contained in CapFuran-FITC under the same experimental and imaging conditions. As can be seen in FIG. 14B, there is negligible penetration of the organoid model by FITC; all of the grey haze depicted is red (the cellular actin channel); there is no green FITC visible anywhere except in sporadic examples of dead cells clinging to the surface, which are known to take up surrounding dye easily. FIG. 15A show pixel intensity plot of the organoid shown in FIG. 14, confirming quantitatively that there is robust, multilayer delivery of FITC via the particle (left side) but only negligible delivery of free FITC via diffusion (black line) in any cell layer, as circumscribed by cellular F-actin (cell cortex) staining (grey line) which indicates where the boundaries of the 3D organoid reside in the height (Z) axis. FIG. 15B shows two different comparative statistical analyses using corrected total cellular fluorescence (CTCF) of the organoids shown in FIG. 14: log of the mean plus or minus 95% confidence interval (left) and log median with box whisker plot (right). A non-parametric Mann Whitney test shows that the very large difference between particle-assisted delivery and free diffusion delivery is highly statistically significant (p<0.001).

Taken together, these data suggest that capsule-mediated CapFuran-FITC delivery can deposit FITC cargo throughout multiple layers of cells, whereas the equivalent amount of free FITC is unable to diffuse through the GAG layer and asymmetric unit membrane plaques to enter cells to any appreciable level. Thus, as also suggested by the experiments in FIG. 12, CapFuran has clear superiority over cargo delivered by free diffusion in this more physiological and formidable human bladder model.

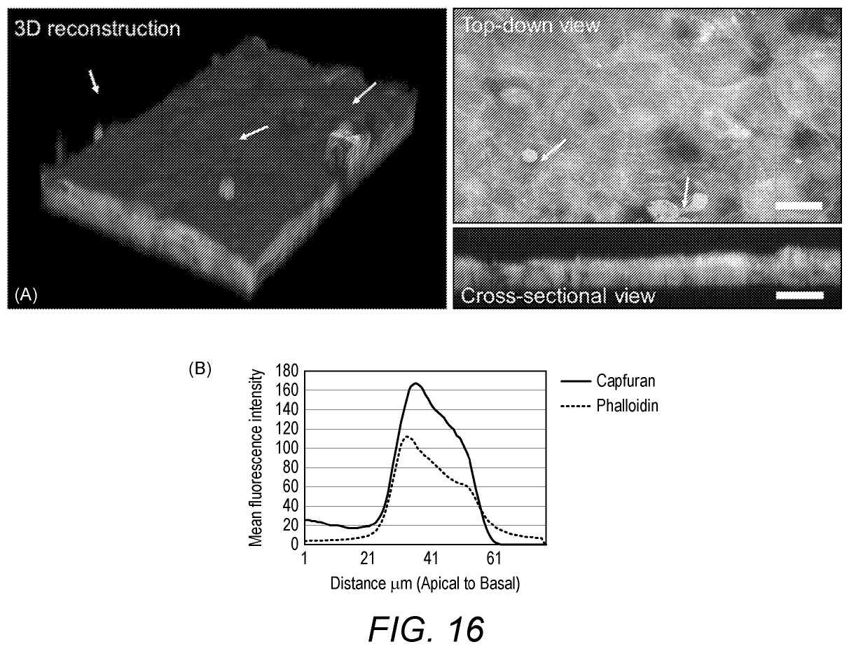

As it is more difficult to track FITC when it becomes distributed and spread out through multiple layers, we performed an experiment similar to that depicted in FIG. 14, except that we used a version of the capsules with FITC distributed in all four layers to make it brighter (CapFuran-FITC-4x [Example Particle 5]). FIG. 16A confirms that this capsule causes robust and deep penetration by FITC (bright grey haze) after 2 hours of treatment in the 3D organoid model; this effect is quantified in 16B with a profile plot showing that FITC (black line) is in the same cellular compartments as cellular F-actin (grey line); that is, intracellular throughout the cell layers.

These experiments taken together show that cargo delivery is highly efficient in a more tissue-like human model and that delivery can occur to layers below the superficial layer of umbrella cells. In contrast, free diffusion appears not to occur to any appreciable level in the more bladder-like organoid, which means that CapFuran has a clear advantage over any strategy that involves distilling free drug intravesically. The ability to penetrate deeply is significant because in the chronic UTI literature, it is reported that in addition to colonization of the apical umbrella cells, the infected bladder also suffers from deeper quiescent intracellular reservoirs further down in the bladder wall, which make total eradication difficult using traditional treatment methods.

CapFuran Efficiently Kills Enterococcus faecalis, a Key Pathogen in Chronic UTI in the Elderly as Well as in Hospital-Acquired Infections.

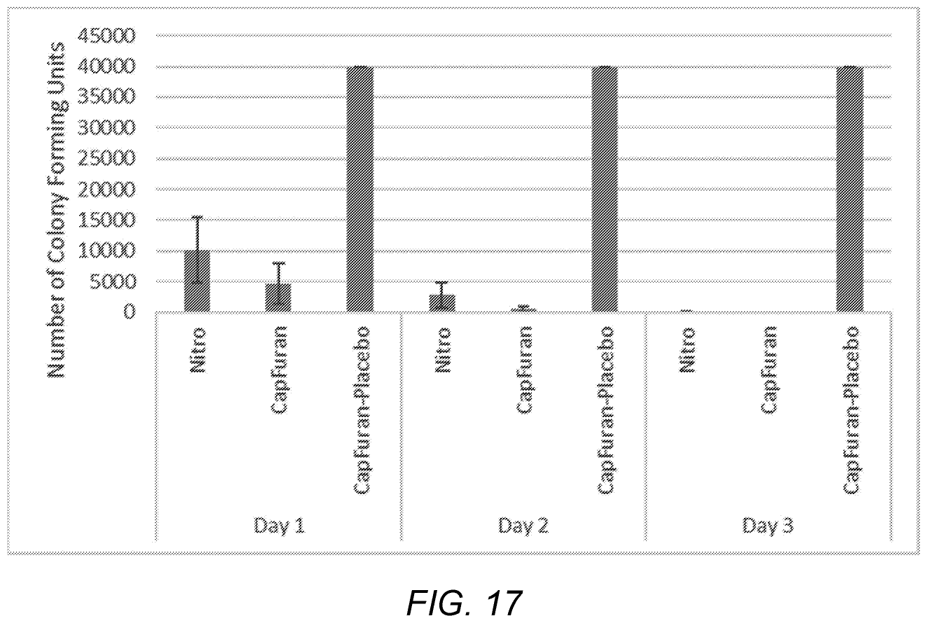

To assess the antimicrobial activity of CapFuran (Example Particle 1), we mixed capsules with E. faecalis bacteria and incubated them over three days, assessing bacteria viability at various time points. FIG. 17 shows data from 6 separate experiments normalised and combined. After one day of treatment, there were significantly fewer colony-forming units (CFU) of viable bacteria for nitrofurantoin (200 ug/mL) and CapFuran (2.0 mg/mL) when compared to the bacteria treated with the CapFuran-Placebo control (Reference Example Particle 6). After the second day there were fewer CFUs than after the first day. After three days of treatment, there were few or no CFUs remaining. The number of CFUs for the CapFuran-Placebo control (Reference Example Particle 6) remained the same for all three days.

These experiments show that there is no significant difference between the killing capability of free nitrofurantoin and CapFuran (Example Particle 1) in a cell-free system, which is important because it shows that encapsulation does not affect the antimicrobial activity of the drug.

CapFuran can Kill Other Bacterial Strains for which Nitrofurantoin is Indicated, Sometimes More Potently than the Equivalent Amount of Free Nitrofurantoin

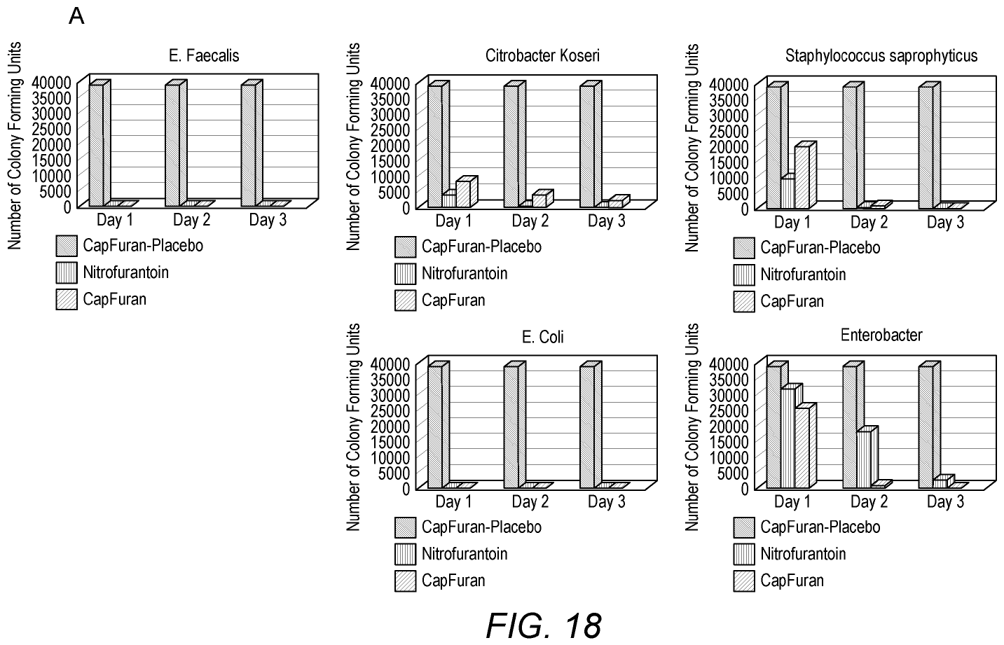

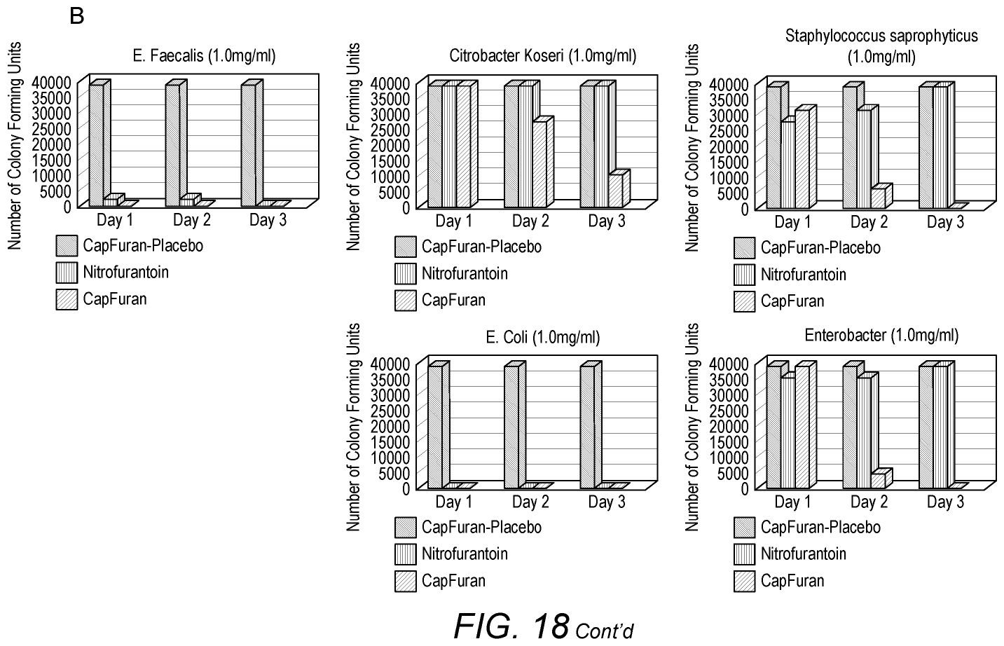

The ability of CapFuran (Example Particle 1) to kill other uropathogens for which nitrofurantoin is indicated was also tested. Along with a normal test strain of E. faecalis, strong killing was also observed in the normal broth assay with CapFuran against E. coli (highly virulent strain UT189), Staphylococcus, Citrobacter, and Enterobacter. As shown in FIG. 18A CapFuran kills as well as free nitrofurantoin in all cases, and far better than free nitrofurantoin in most cases. Specifically, as shown in FIG. 18B, superior CapFuran killing was seen at lower doses with patient-derived strains of Staphylococcus, Enterobacter and Citrobacter. Although there is no lower-dose superiority seen with E. coli, CapFuran is still able to kill this common uropathogen as well as nitrofurantoin at the equivalent dose.

In summary, CapFuran (Example Particle 1) kills as well as the free drug for all species tested. Importantly, CapFuran is able to kill patient derived Staphylococcus, Enterobacter and Citrobacter more efficiently than the equivalent amount of free nitrofuranotin at lower doses. This result is important because, in the case of chronic UTI as an example, sufferers can experience a range of infective species and often more than one organism at a time. For example, while E. faecalis is common in the elderly and prevalent in hospital acquired and catheter-associated UTI, other species also cause UTI. In particular, E. coli is the most common species involved in acute UTI in healthy young women, acquired in the community.

CapFuran can Reduce the Burden of Chronic Infection in the Organoid Model System

The antibiotic protection assay was used to inspect the ability of the CapFuran (Example Particle 1) to enter and kill protected reservoirs. The way the assay works is that E. faecalis bacteria are allowed to infect and invade bladder cells or organoids, before being treated with gentamicin and vancomycin, two antibiotics that cannot penetrate cells and therefore can only kill bacteria on the outside. Afterwards, the cells are challenged with various treatments, and then lysed with detergent and plated to look for any residual bacterial growth. Anything that now grows must have been ‘protected’ by the cell because it was residing inside, and would therefore not have been sensitive to levels achieved in the bladder by non-permeant or poorly permeant oral (conventional) dosing regimes. Bacterial burden was assessed by plating the lysate and counting viable (colony-forming) units.

First, we confirmed that the capsule treatment was not unduly toxic to human cells itself, by using a commercial kit that measures release of the intracellular protein lactate dehydrogenase (LDH), which is a biochemical proxy for cellular damage. As shown in FIG. 19A, human urothelial organoids treated with two different doses of CapFuran exhibited no more toxicity than the equivalent amount of free drug itself; in the case of the higher dose, the capsules were less toxic. Next, we performed the protection assay. As seen in FIG. 19B, cells receiving “mock” treatment have high amounts of intracellular bacteria after lysis. Free nitrofurantoin is able to reduce the number of intracellular bacteria, but CapFuran is approximately ten times more effective, in a manner that is statistically significant (p=0.017). These results show that CapFuran is still able to exert its antibiotic activity inside a cell, emphasizing its utility in intracellular killing. In chronic UTI, for example, intracellular reservoirs are thought to contribute to treatment recalcitrance and recurrence, so that ability to eradicate such reservoirs is an important advance. Most traditional antibiotics are cell impermeant and even if permeant, would not be expected to accumulate to high doses within cells by free diffusion. This experiment also shows that CapFuran is superior to the same amount of free drug in this model system.

Biofilm Disruption

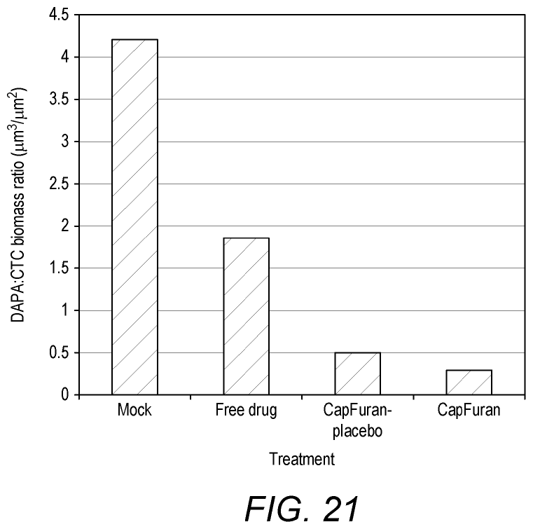

One key anti-biofilm effect of encapsulated drugs is thought to be their ability to disrupt the architecture. Biofilms of E. faecalis were grown and treated with CapFuran-FITC (Example Particle 2) to determine how biomass and structure were affected. As shown in FIG. 20, CapFuran-FITC (Example Particle 2) was able to cause a significant disruption in the biofilm structure above and beyond what was seen with nitrofurantoin alone or with CapFuran-Placebo (Reference Example Particle 6). The increase in red colour exhibited in the CapFuran-FITC (Example Particle 2) condition (not possible to visualize in a black and white photo; see FIG. 21 for quantification) indicates an increase in bacterial respiration, indicating that this biofilm had been roused from its normal dormancy. This is important because antibiotics target the processes of actively dividing cells to exert their antimicrobial effect. The effect of treatment was quantified by comparing bacterial DNA signals to CTC staining as a ratio of biomass. As shown in FIG. 20, while free drug on its own has some effect on biomass, the capsules, even without cargo, cause more disruption (CapFuran-Placebo (Example Reference Particle 6), whereas the strongest effect comes from capsules combined with drug (Example Particle 1: CapFuran). This may be due to rousing of bacteria from dormancy after mechanical disruption by the capsules, which then provides a metabolic target for the antibiotic to act upon.

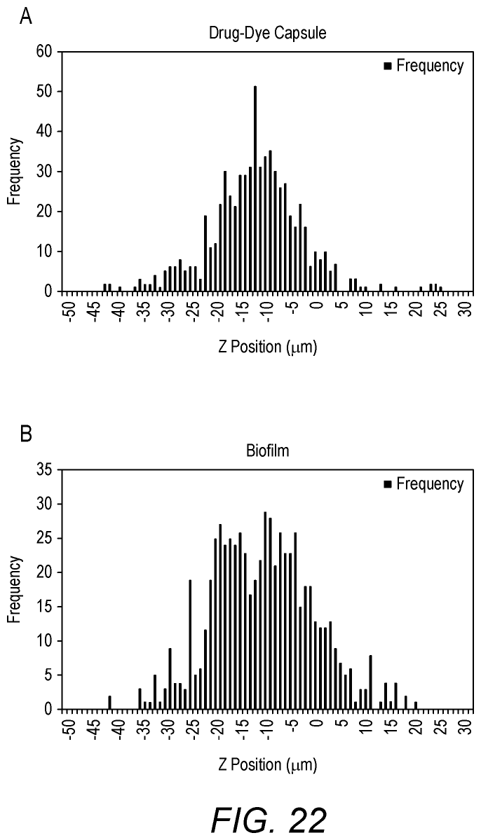

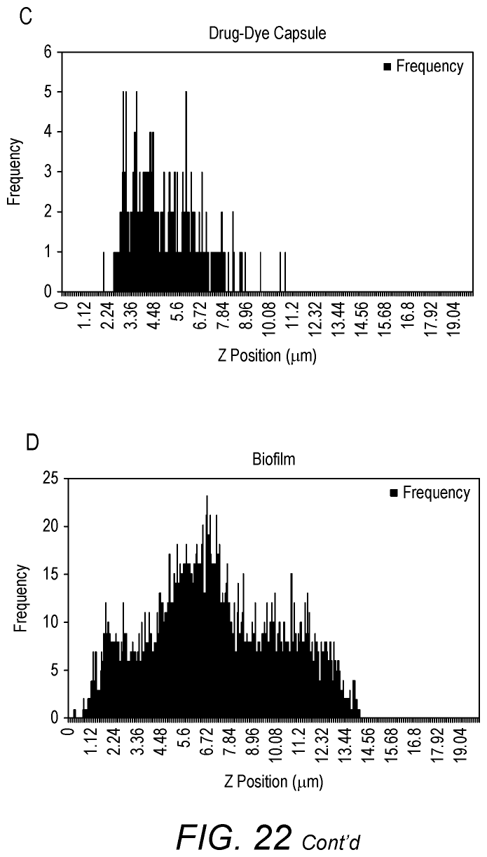

Biofilms are protected by a polymeric capsule which makes the entrance of drugs, including antibiotics, difficult. The ability of CapFuran-FITC (Example Particle 2) to enter biofilms was therefore tested. As shown in FIG. 22, analysis of the distribution of FITC showed that the capsules were present in the centres (HCLT glass slide model) or near to the bottom (PPM model) after treatment, showing robust penetration behaviour.

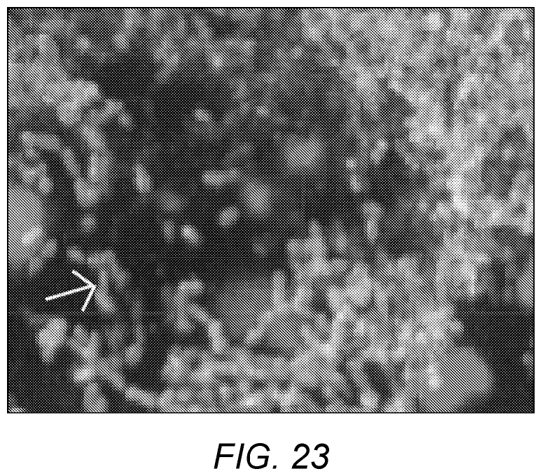

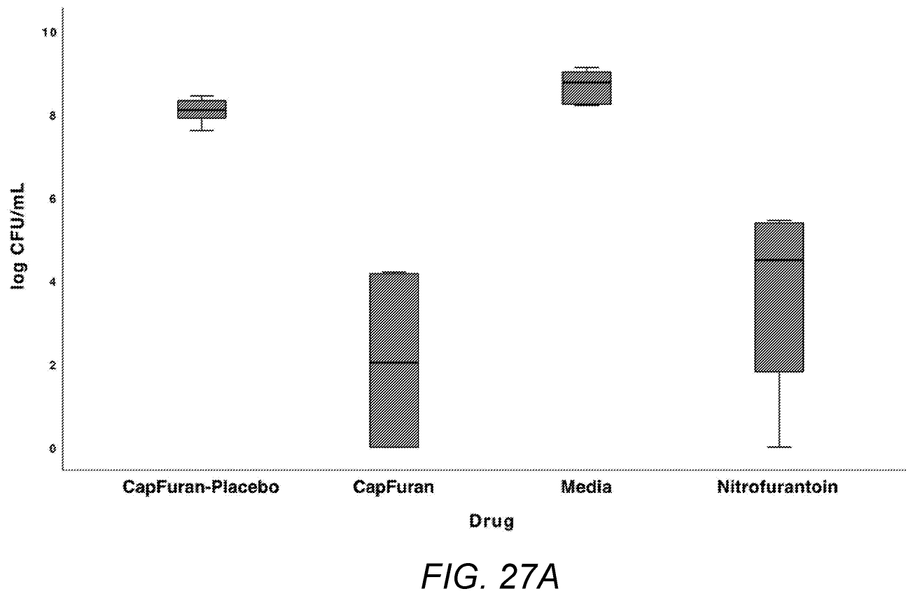

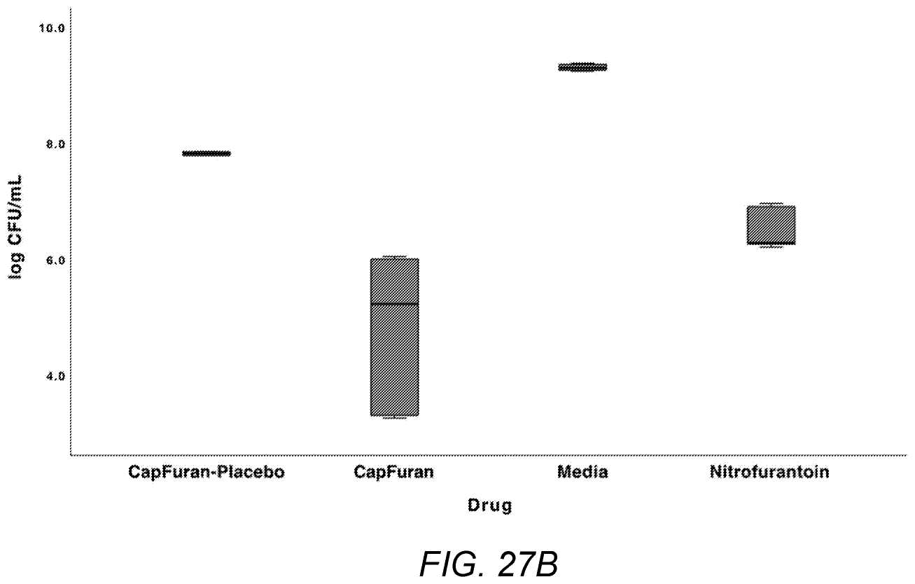

As further proof of biofilm penetration, images at higher magnification after treatment with CapFuran-FITC (Example Particle 2) showed that not only had the green fluorescent cargo been ferried within the biofilm, but the individual bacteria within had taken up the dye (FIG. 23). As shown in FIGS. 27A and 27B CapFuran's ability to penetrate biofilms was mirrored by its ability to treat them. Specifically, FIG. 27A shows that CapFuran performs better than the free drug at killing planktonic bacteria surrounding the biofilm, while FIG. 27B shows that it performs much better than the free drug at killing bacteria incorporated within the solid biofilm.

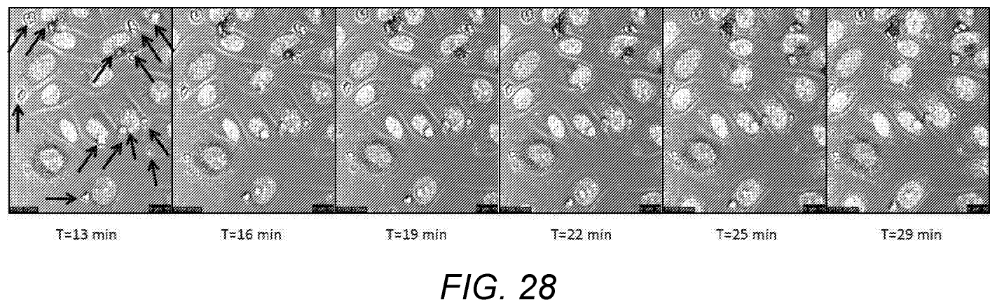

We wanted to understand in more detail how CapFuran delivers cargo in real time, so we performed timelapse videomicroscopy using a confocal microscope. We added 2 mg/ml CapFuran-FITC to HBLAK cells growing in culture and filmed the cells for one hour after addition of the treatment. As shown in FIG. 28, the capsules appear to be docked onto cells as early as 13 minutes post-treatment. They do not actually enter the cells to deliver cargo; instead, they appear to pump cargo into the cell from the outside in a swift and simultaneous fashion, such that at some point between 19 and 22 minutes, pumping begins, whereas the cells are completely full of the maximum amount of cargo by approximately half an hour. This result is significant because the capsules are quite large compared with the cells, and not being incorporated into the cells is a desirable attribute, as they would have no chance to disrupt intracellular function and would also presumably be easier to flush out of the bladder once cargo is delivered.

1. A method for treatment of a disease of the urinary tract, the method comprising administering to a host a composition comprising a particle comprising a biodegradable and hydrolysable polymer, wherein a medicament for the treatment of the disease of the urinary tract is dispersed in the polymer, wherein the particle has a dimension of from 1 μm to 30 μm.

2. A method according to claim 1, wherein the method comprises intracellular delivery of the medicament.

3. A method according to claim 1, wherein the composition is delivered via a synthetic conduit to the urinary tract for the treatment.

4. (canceled)

5. A method according to claim 1, wherein the medicament is an antibiotic.

6. A method according to claim 5, wherein the medicament is selected from nitrofurantoin, norfloxacin, ampicillin, cephalosporins, ceftriaxone, cephalexin, ciprofloxacin, fosfomycin, levofloxacin and trimethoprim/sulfamethoxazole.

7. A method according to claim 1, wherein the disease is bacterial infection.

8. A method according to claim 6, wherein the bacterial infection is selected from E. faecalis, E. coli, Klebsiella, Enterobacter, Staphylococcus and Citrobacter.

9. A method according to claim 1, wherein the disease is a urinary tract infection.

10. A method according to claim 1, wherein the polymer is a polyester or a polyanhydride.

11. A method according to claim 1, wherein the polymer hydrolyses to species selected from alkanoic acids and hydroxyl alkanoic acids.

12. A method according to claim 1, wherein the polymer is selected from poly(lactic-co-glycolic acid) (PLGA), Polyglycolic acid (PGA), Polylactic acid (PLA), Poly (Î-caprolactone) (PCL) and a polyhydroxylalkanoate (PHA).

13. (canceled)

14. A method according to claim 1, wherein the particle further comprises a fluorescent dye, a non-ionic surfactant, or both a fluorescent dye and a non-ionic surfactant.

15. (canceled)

16. A method according to claim 1, wherein the particle has a dimension of 10 μm or less.

17. A method according to claim 1, wherein the composition comprises a plurality of the particles comprising the biodegradable and hydrolysable polymer and the medicament, wherein at least 90%, by number, of the particles comprising the biodegradable and hydrolysable polymer and the medicament have a diameter, as measured using a scanning electron microscope, of 10 μm or less.

18-26. (canceled)

27. A method according to claim 1, wherein the particle is made by electrohydrodynamic processing.

28. A process for producing particles comprising a biodegradable and hydrolysable polymer, the process comprising:
(i) providing an electrohydrodynamic device comprising
at least two concentrically arranged, spaced apart hollow needles, the needles together defining a core channel, and an outer concentrically disposed tubular channel; and
a means for applying a voltage to the needles;
(ii) passing fluid mediums through the hollow core channel, and the outer concentrically disposed tubular channel, wherein at least one of the fluid mediums in one of the channels has therein a biodegradable and hydrolysable polymer and a medicament for the treatment of a disease of the urinary tract; and
(iii) applying a voltage to the needles, such that, on leaving the needles, the particles comprising the biodegradable and hydrolysable polymer are formed, wherein the medicament for the treatment of a disease of the urinary tract is dispersed in the polymer, and at least some of the particles have a dimension of from 1 μm to 30 μm.

29. A process according to claim 28, wherein the device comprises at least four concentrically arranged, spaced apart hollow needles, the needles together defining a core channel, at least two intermediate channels and an outer concentrically disposed tubular channel, and fluid mediums are passed down the core channel, the at least two intermediate channels and an outer concentrically disposed tubular channel, and at least one of the fluid mediums in one of the channels comprises a biodegradable and hydrolysable polymer and a medicament for the treatment of a disease of the urinary tract.

30. A method for treating a disease, the method comprising intracellular delivery of a particle or a medicament from the particle, the particle comprising a biodegradable and hydrolysable polymer, wherein the medicament is dispersed in the polymer, wherein the particle has a dimension of from 1 μm to 30 μm.

31. A method according to claim 30, wherein the method is for the treatment of a cancer of the urinary tract.

32. A method according to claim 30, wherein the method is for the treatment of a biofilm.
